(19) United States
(12) Patent Application Publication
(10) Pub. No.: US 2015/0211028 A1
(43) Pub. Date: Jul. 30, 2015

(54) Production of 1,3-Propanediol in Cyanobacteria

(71) Applicant: Algenol Biofuels Inc., Fort Myers, FL (US)

(72) Inventors: Jonathan Wong CHIN, Fort Myers, FL (US); Matthew Alexander ANDERSON, Estero, FL (US); Jianping CUI, Naples, FL (US); Matt SPIEKER, San Diego, CA (US)

(73) Assignee: ALGENOL BIOFUELS INC., Fort Myers, FL (US)

(21) Appl. No.: 14/681,462

(22) Filed: Apr. 8, 2015

Related U.S. Application Data
(63) Continuation of application No. PCT/US13/65574, filed on Oct. 18, 2013.

(51) Int. Cl. C12P 7/18; C12N 9/16; C12N 9/88; C12N 9/04

(52) U.S. Cl. CPC C12P 7/18; C12N 9/0006; C12N 9/16; C12N 9/88; C12Y 101/01; C12Y 301/03021; C12Y 402/0103; C12Y 101/01202

(57) ABSTRACT

Cyanobacterial host cells are modified to produce useful chemicals such as 1,3-propanediol and glycerol.

### CROSS-REFERENCE TO RELATED APPLICATIONS

This application is a continuation of International Application No. PCT/US13/65574, filed Oct. 18, 2013, which claims the benefit of U.S. Provisional Patent Application Ser. No. 61/715,371, filed Oct. 18, 2012. The disclosures of these documents are incorporated herein by reference in their entirety.

### REFERENCE TO SEQUENCE LISTING

This application contains a sequence listing submitted by EFS-Web, created on Oct. 11, 2013, is named “Propanediol_{—}1_{—}3PCT_seq_list_ST25,” and is 102 KB in size.

### FIELD OF THE INVENTION

The present invention relates to cyanobacterial host cells which are modified to produce useful chemicals, such as 1,3-propanediol.

### BACKGROUND OF THE INVENTION

Cyanobacteria (also known as “blue-green algae”) are small, mainly aquatic, prokaryotic cells that have the ability to perform oxygenic photosynthesis and make biomass and organic compounds from the input of light, nutrients, and CO₂. Cyanobacteria can be genetically enhanced to produce valuable products, such as biofuels, pharmaceuticals, nutraceuticals, etc. For example, the transformation of the cyanobacterial genus Synechococcus with genes that encode specific enzymes that can produce ethanol for biofuel production has been described (U.S. Pat. Nos. 6,699,696 and 6,306,639, both to Woods et al.). The transformation of the cyanobacterial genus Synechocystis is described, for example, in PCT/US2007/001071, PCT/EP2009/000892, and in PCT/EP2009/060526.

The compound 1,3-propanediol is a viscous, colorless, and water-miscible liquid. 1,3-propanediol can be used as a building block for the production of polyethylene terephthalate (PET), nylon, and a PET variant, polytrimethylene terephthalate (PTT). 1,3-propanediol can also be used in a variety of materials, including adhesives, laminates, clothing, carpets, plastics, coatings, moldings, antifreeze, aliphatic polyesters, and copolyesters.

1,3-propanediol may be produced synthetically or by fermentation. Several different methods of making 1,3-propanediol synthetically have been utilized. For instance, 1,3-propanediol may be generated synthetically from 1) ethylene oxide over a catalyst in the presence of phosphine, water, carbon monoxide, hydrogen, and an acid; 2) by the catalytic solution phase hydration of acrolein followed by reduction; or 3) from hydrocarbons (such as glycerol) reacted in the presence of carbon monoxide and hydrogen over catalysts having atoms from Group VIII of the Periodic Table.

U.S. Pat. No. 5,786,524 teaches the preparation of 1,3-propanediol from ethylene oxide. The process involves (1) the cobalt-catalyzed hydroformylation (reaction with synthesis gas, H₂/CO) of ethylene oxide to prepare a dilute solution of intermediate 3-hydroxypropanal (HPA); (2) extraction of the HPA into water to form a more concentrated HPA solution; and (3) hydrogenation of the HPA to propanediol.

U.S. Patent Application Publication No. 20110125118 describes a prophetic example of a method of synthetically producing 1,3-propanediol from acrylic acid. The method involves the hydrogenation of 3-hydroxypropionic acid in a liquid phase (water and cyclohexane), in the presence of an unsupported ruthenium catalyst, using a stirred reactor tank at 1000 psi and 150° C.

1,3-propanediol produced biologically via fermentation of sugars and glycerol using recombinantly-engineered bacteria has been described, for example, in U.S. Pat. No. 5,686,276, U.S. Pat. No. 6,358,716, and U.S. Pat. No. 6,136,576.

U.S. Pat. No. 8,216,816 describes a prophetic example of a biological engineering method that can be used to produce 1,3-propanediol in microorganisms. The prophetic method utilizes the following biological pathway: the enzyme sn-glycerol-3-P dehydrogenase dar1 (EC 1.1.1; derived from S. cerevisiae) generates sn-glycerol-3-P from dihydroxyacetone-P, NADH, and NADPH. The enzyme sn-glycerol-3-phosphatase gpp2 (EC 3.1.3.21; derived from S. cerevisiae) generates glycerol from sn-glycerol-3-P. The enzyme glycerol dehydratase dhaB1-3 (EC 4.2.1.30; derived from K. pneumonia) generates 3-hydroxypropanal from glycerol. The enzyme 1,3-propanediol oxidoreductase dhaT (EC 1.1.1.202; derived from K. pneumonia) converts 3-hydroxypropanal and NADH to 1,3-propanediol.

Current methods of producing 1,3-propanediol require the input of an organic carbon source, such as fossil fuel or sugar. An object of the invention is a method of producing these compounds from CO₂ as the input carbon source, rather than from fossil fuels or from other organic starting materials.

### SUMMARY OF THE INVENTION

In an aspect of the invention, a genetically enhanced nucleic acid sequence for the production of 1,3-propanediol in cyanobacteria is provided, having at least one promoter capable of regulating gene expression in cyanobacteria, and the genes DAR1, GPP2, dhaB1-3, orfZ, orf2b, and yqhD. The nucleic acid sequence can be capable of replicating in a cyanobacterial cell. At least one of the genes can be present on a plasmid, such as an exogenously derived or endogenously derived plasmid, or it may be present on the cyanobacterial chromosome. The promoter can be, for example, Psrp, PnblA₇₁₂₀, PrbcL₆₈₀₃, PsmtA₇₀₀₂, and ziaR-PziaA₆₈₀₃. In an embodiment, the promoter sequence can be, for example, SEQ ID NO: 1, SEQ ID NO: 2, SEQ ID NO: 3, SEQ ID NO: 4, or SEQ ID NO: 5.

The gene encoding DAR1 can have at least 98% identity to SEQ ID NO: 6. The DAR1 polypeptide can have at least 98% identity to SEQ ID NO: 7. The gene encoding GPP2 can have, for example, at least 98% identity to SEQ ID NO: 8. The GPP2 polypeptide can have at least 98% identity to SEQ ID NO: 9. The dhaB1-3-encoding nucleic acid sequence can have at least 98% identity to SEQ ID NO: 10. The dhaB1-3 nucleic acid sequence can encode three separate polypeptides, DhaB1, DhaB2, and DhaB3, where the DhaB1 polypeptide can have at least 98% sequence identity to SEQ ID NO: 12; the DhaB2 polypeptide can have at least 98% identity to SEQ ID NO: 14; and the DhaB3 polypeptide can have at least 98% identity to SEQ ID NO: 16. The orfZ and orf2b nucleic acid sequences can have at least 98% identity to SEQ ID NO: 17. The orfZ gene can encode a polypeptide having at least 98% identity to SEQ ID NO: 19, and wherein the orf2b gene can encode a polypeptide having at least 98% identity to SEQ ID NO: 21. The yqhD gene can have at least 98% identity to SEQ ID NO: 22. The YqhD polypeptide can have at least 98% identity to SEQ ID NO: 23.

In another aspect of the invention, a genetically enhanced cyanobacterial cell having a DAR1 gene, a GPP2 gene, a nucleic acid sequence of the dhaB1-3 genes, an orfZ gene, an orf2b gene, and a yqhD gene is provided, where the cell produces 1,3-propanediol. The cyanobacterium can be, for example, Synechocystis sp. PCC 6803 or Synechococcus sp. PCC 7002.

In another aspect of the invention, a method of producing 1,3-propanediol in a cyanobacterial cell is provided, by introducing a nucleic acid sequence having a gene encoding a DAR1 enzyme, a gene encoding a GPP2 enzyme, a gene encoding the DhaB1-3 enzymes, a gene encoding an OrfZ enzyme, a gene encoding an Orf2b enzyme, and a gene encoding a YqhD enzyme to a cyanobacterial cell; and then culturing the cell under conditions which produce 1,3-propanediol.

### BRIEF DESCRIPTION OF THE DRAWINGS

FIG. 1 is a diagram of the biosynthetic pathway used to produce 1,3-propanediol from the central carbon metabolite glycerone phosphate (DHAP). When genes from this pathway are transferred to cyanobacteria, these metabolites can be produced through photosynthetic and gluconeogenic pathways using CO₂ as the input carbon source.

FIG. 2 is a linear diagram of the genes and relevant features in the broad host range RSF1010-derivative plasmid pSL1211, which was used as the basis for the expression vectors described herein. Relevant restriction sites and terminator regions are indicated.

FIG. 3 is a linearized map of the pSL1211-derived plasmid (“pABb”) that was used as the framework plasmid for the insertion of the propanediol genes described in Example 4. The promoter, terminator (TT), and ribosomal binding site (RBS) are indicated.

FIG. 4 is a linearized map of the nucleic acid segment containing the coding region for the genes involved in the production of 1,3-propanediol, as described in Example 4. The location of genes GPD1 (“DAR1”), HOR2 (“GPP2”), the dhaB1-3 genes, an orfZ gene, an orf2b gene, and a yqhD gene are indicated.

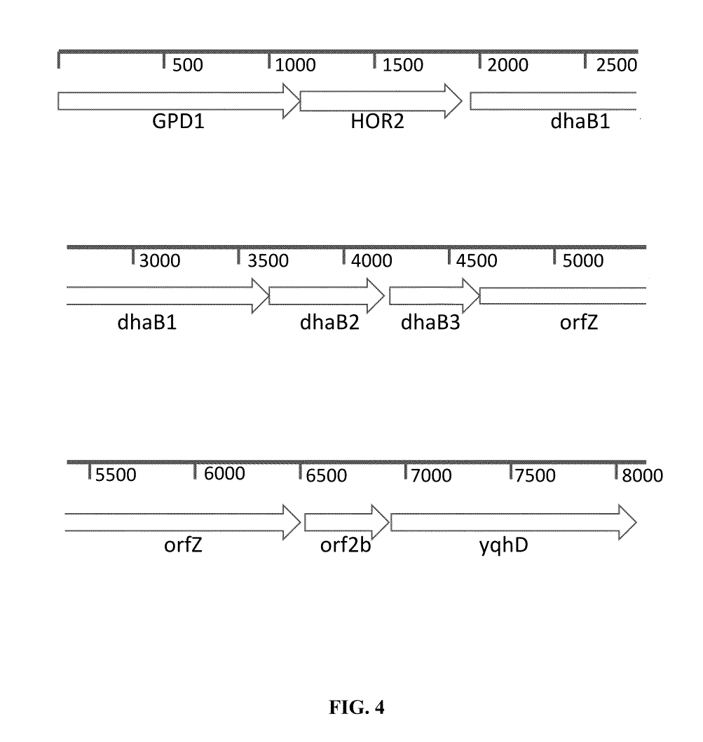

FIG. 5 is an overlay of chromatograph traces confirming the successful transformation and gene expression of the initial portion of the 1,3-propanediol pathway, from glycerone phosphate to glycerol. The trace shows that Synechocystis sp. PCC 6803, harboring plasmid pAB1001, is capable of producing the intermediate glycerol. Also shown are Synechocystis sp. PCC 6803 wild type, and a 100 μM glycerol standard. The traces were produced from a separation of glycerol using liquid chromatography on a Dionex system. The peak having a retention time of 8.1 minutes was identified as glycerol.

FIG. 6 is a graph of a 5× concentrated methanol/phosphate extract from Synechococcus sp. PCC 7002 harboring the plasmid pAB1003, which was given a glycerol input feed as described in Example 8. The trace was produced from a separation of 1,3-propanediol using gas chromatography. Peaks were identified using mass spectroscopy. The peak having a retention time of 5.88 minutes was identified as 1,3-propanediol. This peak was not present in wild type Synechococcus sp. PCC 7002.

### DETAILED DESCRIPTION

Cyanobacterial host cells can be genetically enhanced in order to produce various valuable chemical products, such as 1,3-propanediol. In an embodiment, genes involved in the biosynthetic pathways for 1,3-propanediol can be transferred to a cyanobacterial host cell. The inserted heterologous genes can be present on extrachromosomal plasmids, or they can be present on the cyanobacterial chromosome. The cyanobacterial cells are then cultured following general cyanobacterial methods, and the propanediol is removed at the appropriate time. The production of 1,3-propanediol in cyanobacteria rather than by use of chemical means allows the compounds to be produced from carbon dioxide as the initial carbon source, rather than from crude oil or other organic carbon sources.

Aspects of the invention utilize techniques and methods common to the fields of molecular biology, microbiology and cell culture. Useful laboratory references for these types of methodologies are readily available to those skilled in the art. See, for example, Molecular Cloning: A Laboratory Manual (Third Edition), Sambrook, J., et al. (2001) Cold Spring Harbor Laboratory Press; Current Protocols in Microbiology (2007) Edited by Coico, R, et al., John Wiley and Sons, Inc.; The Molecular Biology of Cyanobacteria (1994) Donald Bryant (Ed.), Springer Netherlands; Handbook Of Microalgal Culture Biotechnology And Applied Phycology (2003) Richmond, A.; (ed.), Blackwell Publishing; and “The cyanobacteria, molecular Biology, Genomics and Evolution”, Edited by Antonia Herrero and Enrique Flores, Caister Academic Press, Norfolk, UK, 2008.

All publications and patent applications mentioned in this specification are herein incorporated by reference to the same extent as if each individual publication or patent application was specifically and individually indicated to be incorporated by reference.

### DEFINITIONS

Unless defined otherwise, all technical and scientific terms used herein have the meaning commonly understood by a person skilled in the art to which this invention belongs. As used herein, the following terms have the meanings ascribed to them unless specified otherwise.

The term “about” is used herein to mean approximately, in the region of, roughly, or around. When the term “about” is used in conjunction with a numerical value/range, it modifies that value/range by extending the boundaries above and below the numerical value(s) set forth. In general, the term “about” is used herein to modify a numerical value(s) above and below the stated value(s) by a variance of 20%.

The term “Cyanobacterium” refers to a member from the group of photoautotrophic prokaryotic microorganisms which can utilize solar energy and fix carbon dioxide. Cyanobacteria are also referred to as blue-green algae.

The terms “host cell” and “recombinant host cell” are intended to include a cell suitable for metabolic manipulation, e.g., which can incorporate heterologous polynucleotide sequences, e.g., which can be transformed. The term is intended to include progeny of the cell originally transformed. In particular embodiments, the cell is a prokaryotic cell, e.g., a cyanobacterial cell. The term recombinant host cell is intended to include a cell that has already been selected or engineered to have certain desirable properties and to be suitable for further enhancement using the compositions and methods of the invention.

“Competent to express” refers to a host cell that provides a sufficient cellular environment for expression of endogenous and/or exogenous polynucleotides.

As used herein, the term “genetically enhanced” refers to any change in the endogenous genome of a wild type cell or to the addition of non-endogenous genetic code to a wild type cell, e.g., the introduction of a heterologous gene. More specifically, such changes are made by the hand of man through the use of recombinant DNA technology or mutagenesis. The changes can involve protein coding sequences or non-protein coding sequences such as regulatory sequences as promoters or enhancers.

“Polynucleotide” and “nucleic acid” refer to a polymer composed of nucleotide units (ribonucleotides, deoxyribonucleotides, related naturally occurring structural variants, and synthetic non-naturally occurring analogs thereof) linked via phosphodiester bonds, related naturally occurring structural variants, and synthetic non-naturally occurring analogs thereof. Thus, the term includes nucleotide polymers in which the nucleotides and the linkages between them include non-naturally occurring synthetic analogs. It will be understood that, where required by context, when a nucleotide sequence is represented by a DNA sequence (i.e., A, T, G, C), this also includes an RNA sequence (i.e., A, U, G, C) in which “U” replaces “T.”

The nucleic acids of this present invention may be modified chemically or biochemically or may contain non-natural or derivatized nucleotide bases, as will be readily appreciated by those of skill in the art. Such modifications include, for example, labels, methylation, substitution of one or more of the naturally occurring nucleotides with an analog, internucleotide modifications such as uncharged linkages, charged linkages, alkylators, intercalators, pendent moieties, modified linkages, and chelators. Also included are synthetic molecules that mimic polynucleotides in their ability to bind to a designated sequence via hydrogen bonding and other chemical interactions.

The term “nucleic acid” (also referred to as polynucleotide) is also intended to include nucleic acid molecules having an open reading frame encoding a polypeptide, and can further include non-coding regulatory sequences and introns. In addition, the terms are intended to include one or more genes that map to a functional locus. In addition, the terms are intended to include a specific gene for a selected purpose. The gene can be endogenous to the host cell or can be recombinantly introduced into the host cell.

In one aspect the invention also provides nucleic acids which are at least 60%, 70%, 80% 90%, 95%, 97%, 98%, 99%, or 99.5% identical to the nucleic acids disclosed herein.

The percentage of identity of two nucleic acid sequences or two amino acid sequences can be determined using the algorithm of Thompson et al. (CLUSTALW, 1994, Nucleic Acids Research 22: 4673-4680). A nucleotide sequence or an amino acid sequence can also be used as a so-called “query sequence” to perform a search against public nucleic acid or protein sequence databases in order, for example, to identify further unknown homologous promoters, which can also be used in embodiments of this invention. In addition, any nucleic acid sequences or protein sequences disclosed in this patent application can also be used as a “query sequence” in order to identify yet unknown sequences in public databases, which can encode for example new enzymes, which could be useful in this invention. Such searches can be performed using the algorithm of Karlin and Altschul (1990, Proceedings of the National Academy of Sciences U.S.A. 87: 2,264 to 2,268), modified as in Karlin and Altschul (1993, Proceedings of the National Academy of Sciences U.S.A. 90: 5,873 to 5,877). Such an algorithm is incorporated in the NBLAST and XBLAST programs of Altschul et al. (1990, Journal of Molecular Biology 215: 403 to 410). Suitable parameters for these database searches with these programs are, for example, a score of 100 and a word length of 12 for BLAST nucleotide searches as performed with the NBLAST program. BLAST protein searches are performed with the XBLAST program with a score of 50 and a word length of 3. Where gaps exist between two sequences, gapped BLAST is utilized as described in Altschul et al. (1997, Nucleic Acids Research, 25: 3,389 to 3,402).

A “promoter” is a nucleic acid control sequence that directs transcription of an associated polynucleotide, which may be a heterologous polynucleotide or a native polynucleotide. A promoter includes nucleic acid sequences near the start site of transcription, such as a polymerase binding site. The promoter also optionally includes distal enhancer or repressor elements which can be located as much as several thousand base pairs from the start site of transcription. In one embodiment, the transcriptional control of a promoter results in an increase in expression of the gene of interest. In another embodiment, a promoter is placed 5′ to the gene-of-interest.

A promoter can be used to replace the natural promoter, or can be used in addition to the natural promoter. A promoter can be endogenous with regard to the host cell in which it is used or it can be a heterologous polynucleotide sequence introduced into the host cell, e.g., exogenous with regard to the host cell in which it is used. A promoter can also be endogenous with regard to the host cell, but derived from a different original gene. In an embodiment, the promoter is a constitutive promoter. In another embodiment, the promoter is inducible, meaning that certain exogenous stimuli (e.g., nutrient starvation, heat shock, mechanical stress, light exposure, etc.) will induce the promoter leading to the transcription of the gene.

The term “recombinant nucleic acid molecule” includes a nucleic acid molecule (e.g., a DNA molecule) that has been altered, modified or engineered such that it differs in nucleotide sequence from the native or natural nucleic acid molecule from which the recombinant nucleic acid molecule was derived (e.g., by addition, deletion or substitution of one or more nucleotides). The recombinant nucleic acid molecule (e.g., a recombinant DNA molecule) can also refer to a nucleic acid that originated in a different location on the DNA, or from a different organism.

“Recombinant” refers to polynucleotides synthesized or otherwise manipulated in vitro (“recombinant polynucleotides”) and to methods of using recombinant polynucleotides to produce gene products encoded by those polynucleotides in cells or other biological systems. For example, a cloned polynucleotide may be inserted into a suitable expression vector, such as a bacterial plasmid, and the plasmid can be used to transform a suitable host cell. In an embodiment, the recombinant polynucleotide can be located on an extrachromosomal plasmid. In another embodiment, the recombinant nucleic acid can be located on the cyanobacterial chromosome. A host cell that comprises the recombinant polynucleotide is referred to as a “recombinant host cell” or a “recombinant bacterium” or a “recombinant cyanobacterium.” The gene is then expressed in the recombinant host cell to produce, e.g., a “recombinant protein.” A recombinant polynucleotide may serve a non-coding function (e.g., promoter, origin of replication, ribosome-binding site, etc.) as well.

The term “homologous recombination” refers to the process of recombination between two nucleic acid molecules based on nucleic acid sequence similarity. The term embraces both reciprocal and nonreciprocal recombination (also referred to as gene conversion). In addition, the recombination can be the result of equivalent or non-equivalent cross-over events. Equivalent crossing over occurs between two equivalent sequences or chromosome regions, whereas nonequivalent crossing over occurs between identical (or substantially identical) segments of nonequivalent sequences or chromosome regions. Unequal crossing over typically results in gene duplications and deletions. For a description of the enzymes and mechanisms involved in homologous recombination see Watson et al., “Molecular Biology of the Gene,” pages 313-327, The Benjamin/Cummings Publishing Co. 4th ed. (1987).

The term “non-homologous or random integration” refers to any process by which DNA is integrated into the genome that does not involve homologous recombination. It appears to be a random process in which incorporation can occur at any of a large number of genomic locations.

The term “expressed endogenously” refers to polynucleotides that are native to the host cell and are naturally expressed in the host cell.

The term “operably linked” refers to a functional relationship between two parts in which the activity of one part (e.g., the ability to regulate transcription) results in an action on the other part (e.g., transcription of the sequence). Thus, a polynucleotide is “operably linked to a promoter” when there is a functional linkage between a polynucleotide expression control sequence (such as a promoter or other transcription regulation sequences) and a second polynucleotide sequence (e.g., a native or a heterologous polynucleotide), where the expression control sequence directs transcription of the polynucleotide. The nucleotide sequence of the nucleic acid molecule or gene of interest is linked to the regulatory sequence(s) in a manner which allows for regulation of expression (e.g., enhanced, increased, constitutive, basal, attenuated, decreased or repressed expression) of the nucleotide sequence and expression of a gene product encoded by the nucleotide sequence (e.g., when the recombinant nucleic acid molecule is included in a recombinant vector, as defined herein, and is introduced into a microorganism).

The term “vector” as used herein is intended to refer to a nucleic acid molecule capable of transporting another nucleic acid to which it has been linked. One type of vector is a “plasmid,” which generally refers to a circular double stranded DNA molecule into which additional DNA segments may be ligated, but also includes linear double-stranded molecules such as those resulting from amplification by the polymerase chain reaction (PCR) or from treatment of a circular plasmid with a restriction enzyme.

Certain vectors are capable of autonomous replication in a host cell into which they are introduced (e.g., vectors having an origin of replication which functions in the host cell). Other vectors can be integrated into the genome of a host cell upon introduction into the host cell, and are thereby replicated along with the host genome. Moreover, certain vectors are capable of directing the expression of genes to which they are operatively linked. Such vectors are referred to herein as “recombinant expression vectors” (or simply “expression vectors”).

In an embodiment, the RSF1010 vector (Mermet-Bouvier et al., 1993, Current Microbiology 27:323-327), originally derived from E. coli, is used as a base plasmid for expression of the propanediol genes in cyanobacterial host cells. This vector appears to be relatively stable and can exist in the cell at a copy number of about 15-20 per cell.

Other plasmids, such as plasmids derived from an endogenous vector of the host cell strain or another cyanobacterial cell, may also be used. An “endogenous vector” or “endogenous plasmid” refers to an extrachromosomal, circular nucleic acid molecule that is derived from the host cell organism.

The term “gene” refers to an assembly of nucleotides that encode a polypeptide, and includes cDNA and genomic DNA nucleic acids. “Gene” also refers to a nucleic acid fragment that expresses a specific protein or polypeptide, including regulatory sequences preceding (5′ non-coding sequences) and following (3′ non-coding sequences) the coding sequence.

The term “endogenous gene” refers to a native gene in its natural location in the genome of an organism. A “foreign” gene or “heterologous” gene refers to a gene not normally found in the host organism, but that is introduced into the host organism by gene transfer. Foreign genes can comprise native genes inserted into a non-native organism, or chimeric genes. A “transgene” is a gene that has been introduced into the genome by a transformation procedure.

The term “nucleic acid fragment” will be understood to mean a nucleotide sequence of reduced length relative to the reference nucleic acid and comprising, over the common portion, a nucleotide sequence substantially identical to the reference nucleic acid. Such a nucleic acid fragment according to the invention may be, where appropriate, included in a larger polynucleotide of which it is a constituent. Such fragments comprise, or alternatively consist of, oligonucleotides ranging in length from at least about 6 to about 2200 or more consecutive nucleotides of a polynucleotide according to the invention.

The term “open reading frame,” abbreviated as “ORF,” refers to a length of nucleic acid sequence, either DNA, cDNA or RNA, that comprises a translation start signal or initiation codon, such as an ATG or AUG, and a termination codon and can be potentially translated into a polypeptide sequence.

The term “upstream” refers to a nucleotide sequence that is located 5′ to reference nucleotide sequence. In particular, upstream nucleotide sequences generally relate to sequences that are located on the 5′ side of a coding sequence or starting point of transcription. For example, most promoters are located upstream of the start site of transcription.

The term “downstream” refers to a nucleotide sequence that is located 3′ to reference nucleotide sequence. In particular, downstream nucleotide sequences generally relate to sequences that follow the starting point of transcription. For example, the translation initiation codon of a gene is located downstream of the start site of transcription.

The term “homology” refers to the percent of identity between two polynucleotide or two polypeptide moieties. The correspondence between the sequence from one moiety to another can be determined by techniques known to the art. For example, homology can be determined by a direct comparison of the sequence information between two polypeptide molecules by aligning the sequence information and using readily available computer programs. Alternatively, homology can be determined by hybridization of polynucleotides under conditions that form stable duplexes between homologous regions, followed by digestion with single-stranded-specific nuclease(s) and size determination of the digested fragments.

As used herein, “substantially similar” refers to nucleic acid fragments wherein changes in one or more nucleotide bases results in substitution of one or more amino acids, but do not affect the functional properties of the protein encoded by the DNA sequence.

The term “substantially similar” also refers to modifications of the nucleic acid fragments of the instant invention such as deletion or insertion of one or more nucleotide bases that do not substantially affect the functional properties of the resulting transcript.

The terms “restriction endonuclease” and “restriction enzyme” refer to an enzyme that binds and cuts within a specific nucleotide sequence within double stranded DNA.

The term “primer” is an oligonucleotide that hybridizes to a target nucleic acid sequence to create a double stranded nucleic acid region that can serve as an initiation point for DNA synthesis under suitable conditions. Such primers may be used in a polymerase chain reaction.

The term “polymerase chain reaction,” also termed “PCR,” refers to an in vitro method for enzymatically amplifying specific nucleic acid sequences. PCR involves a repetitive series of temperature cycles with each cycle comprising three stages: denaturation of the template nucleic acid to separate the strands of the target molecule, annealing a single stranded PCR oligonucleotide primer to the template nucleic acid, and extension of the annealed primer(s) by DNA polymerase. PCR provides a means to detect the presence of the target molecule and, under quantitative or semi-quantitative conditions, to determine the relative amount of that target molecule within the starting pool of nucleic acids.

The term “expression” as used herein refers to the transcription and stable accumulation mRNA derived from a nucleic acid or polynucleotide. Expression may also refer to translation of mRNA into a protein or polypeptide.

An “expression cassette” or “expression construct” refers to a series of polynucleotide elements that permit transcription of a gene in a host cell. Typically, the expression cassette includes a promoter and one or more heterologous or native polynucleotide sequences that are transcribed. Expression cassettes or constructs may also include, e.g., transcription termination signals, polyadenylation signals, and enhancer elements.

The term “codon” refers to a triplet of nucleotides coding for a single amino acid.

The term “codon-anticodon recognition” refers to the interaction between a codon on an mRNA molecule and the corresponding anticodon on a tRNA molecule.

The term “codon bias” refers to the fact that not all codons are used equally frequently in the genes of a particular organism.

The term “codon optimization” refers to the modification of at least some of the codons present in a heterologous gene sequence from a triplet code that is not generally used in the host organism to a triplet code that is more common in the particular host organism. This can result in a higher expression level of the gene of interest.

The expression constructs can be designed taking into account such properties as codon usage frequencies of the organism in which the recombinant genes are to be expressed. Codon usage frequencies can be determined using known methods (see, e.g., Nakamura et al. Nucl. Acids Res. 28:292, 2000). Codon usage frequency tables, including those for cyanobacteria, are also available in the art (e.g., in codon usage databases of the Department of Plant Genome Research, Kazusa DNA Research Institute (www.kazusa.or.jp/codon).

The term “transformation” is used herein to mean the insertion of heterologous genetic material into the host cell. Typically, the genetic material is DNA on a plasmid vector, but other means can also be employed. General transformation methods and selectable markers for bacteria and cyanobacteria are known in the art (Wirth, Mol Gen Genet. 216:175-177 (1989); Koksharova, Appl Microbiol Biotechnol 58:123-137 (2002); Sambrook et al, supra).

The term “selectable marker” means an identifying factor, usually an antibiotic or chemical resistance gene, that is able to be selected for based upon the marker gene's effect, i.e., resistance to an antibiotic, resistance to a herbicide, colorimetric markers, enzymes, fluorescent markers, and the like, wherein the effect is used to track the inheritance of a nucleic acid of interest and/or to identify a cell or organism that has inherited the nucleic acid of interest. Examples of selectable marker genes known and used in the art include: genes providing resistance to ampicillin, streptomycin, gentamycin, spectinomycin, kanamycin, hygromycin, and the like.

A “polypeptide” is a polymeric compound comprised of covalently linked amino acid residues. A “protein” is a polypeptide that performs a structural or functional role in a living cell.

The invention also provides amino acid sequences of the enzymes involved 1,3-propanediol formation, which are at least 60%, 70%, 80%, 85%, 90%, 95%, 97%, 98%, 99%, or 99.5% identical to the amino acid sequences disclosed herein.

The EC numbers cited throughout this patent application are enzyme commission numbers. This is a numerical classification scheme for enzymes based on the chemical reactions which are catalyzed by the enzymes.

A “heterologous gene” refers to a gene that is not naturally present in the cell. Similarly, the term “heterologous nucleic acid” refers to a nucleic acid sequence that is not normally present in the cell.

A “heterologous protein” refers to a protein not naturally produced in the cell.

An “isolated polypeptide” or “isolated protein” is a polypeptide or protein that is substantially free of those compounds that are normally associated therewith in its natural state (e.g., other proteins or polypeptides, nucleic acids, carbohydrates, lipids).

The term “polypeptide fragment” of a polypeptide refers to a polypeptide whose amino acid sequence is shorter than that of the reference polypeptide. Such fragments of a polypeptide according to the invention may have a length of at least about 2 to about 750 or more amino acids.

A “variant” of a polypeptide or protein is any analogue, fragment, derivative, or mutant which is derived from a polypeptide or protein and which retains at least one biological property of the polypeptide or protein. Different variants of the polypeptide or protein may exist in nature. These variants may be allelic variations characterized by differences in the nucleotide sequences of the structural gene coding for the protein, or may involve differential splicing or post-translational modification. The skilled artisan can produce variants having single or multiple amino acid substitutions, deletions, additions, or replacements.

### Preparation of Recombinant Vectors for Genetic Modification of Cyanobacteria

Cyanobacteria can be modified to add enzymatic pathways of interest as shown herein in order to produce 1,3-propanediol. The DNA sequences encoding the genes described herein can be amplified by polymerase chain reaction (PCR) using specific primers. The amplified PCR fragments can be digested with the appropriate restriction enzymes and can then be cloned into either a self-replicating plasmid or an integrative plasmid.

In an embodiment, the nucleic acids of interest can be amplified from nucleic acid samples using amplification techniques. PCR can be used to amplify the sequences of the genes directly from mRNA, from cDNA, from genomic libraries or cDNA libraries. PCR and other in vitro amplification methods may also be useful, for example, to clone nucleic acid sequences that code for proteins to be expressed, to make nucleic acids to use as probes for detecting the presence of the desired mRNA in samples, and for nucleic acid sequencing.

In order to use isolated sequences in the above techniques, recombinant DNA vectors suitable for transformation of cyanobacteria can be prepared. Techniques for transformation are well known and described in the technical and scientific literature. For example, a DNA sequence encoding one or more of the genes described herein can be combined with transcriptional and other regulatory sequences which will direct the transcription of the sequence from the gene in the transformed cyanobacteria.

In an embodiment, an antibiotic resistance cassette for selection of positive clones can be present on the plasmid to aid in selection of transformed cells. For example, genes conferring resistance to ampicillin, gentamycin, kanamycin, or other antibiotics can be inserted into the vector, under the control of a suitable promoter. Other antibiotic resistance genes can be used if desired. In some embodiments, the vector contains more than one antibiotic resistance gene. The presence of a foreign gene encoding antibiotic resistance can be selected, for example, by placing the putative transformed cells into a suitable amount of the corresponding antibiotic, and picking the cells that survive.

In an embodiment, the genes of interest are inserted into the cyanobacterial chromosome. When the cell is polyploid, the gene insertions can be present in all of the copies of the chromosome, or in some of the copies of the chromosome.

In another embodiment, the inserted genes are present on an extrachromosomal plasmid. The extrachromosomal plasmids can be present in a high number or a low number within the genetically enhanced cyanobacterium.

The extrachromosomal plasmid can be derived from an outside source, such as, for example, RSF1010-based plasmid vectors, or it can be derived from an endogenous plasmid from the cyanobacterial cell or from another species of cyanobacteria.

Many cyanobacterial species harbor endogenous vectors that can be used to carry production genes. The cyanobacterium Synechococcus PCC 7002, for example, contains six endogenous plasmids having different numbers of copies in the cyanobacterial cell (Xu et al.: “Expression of genes in cyanobacteria: Adaption of Endogenous Plasmids as platforms for High-Level gene Expression in Synechococcus PCC 7002”, Photosynthesis Research Protocols, Methods in Molecular Biology, 684, pages 273 to 293 (2011)). The endogenous plasmid pAQ1 is present in a number of 50 copies per cell (high-copy), the plasmid pAQ3 with 27 copies, the plasmid pAQ4 with 15 copies and the plasmid pAQ5 with 10 copies per cell (low-copy). In an embodiment, these endogenous plasmids can be used as an integration platform for the 1,3-propanediol genes described herein. The propanediol pathway genes can be integrated into the endogenous cyanobacterial plasmids via homologous recombination, or by other suitable means. It is also possible to create a “shuttle vector” based on the backbone of an endogenous vector, in combination with portions of self-replicating E. coli vectors, for ease of genetic manipulation. Such vectors can be easily manipulated in E. coli, for example, then the vectors can be transferred to the cyanobacterial host strain for the production of 1,3-propanediol or glycerol.

In an embodiment, the inserted genes are present on an extrachromosomal plasmid, wherein the plasmid has multiple copies per cell. The plasmid can be present, for example, at about 1, 3, 5, 8, 10, 15, 20, 30, 40, 50, 60, 70, 80, 90, or more copies per host cyanobacterial cell. In an embodiment, the plasmids are fully segregated.

In another embodiment, the inserted genes are present on one cassette driven by one promoter. In another embodiment, the inserted genes are present on separate plasmids, or on different cassettes.

In another embodiment, the inserted genes are modified for optimal expression by modifying the nucleic acid sequence to accommodate the cyanobacterial cell's protein translation system. Modifying the nucleic acid sequences in this manner can result in an increased expression of the genes.

The inserted genes can be regulated by one promoter, or they can be regulated by individual promoters. The promoters can be constitutive or inducible. The promoter sequences can be derived, for example, from the host cell, from another organism, or can be synthetically derived.

Any desired promoter can be used to regulate the expression of the genes for 1,3-propanediol production. Exemplary promoter types include but are not limited to, for example, constitutive promoters, inducible promoters (e.g., by nutrient starvation, heat shock, mechanical stress, environmental stress, metal concentration, light exposure, etc.), endogenous promoters, heterologous promoters, and the like.

In an embodiment, the inserted genes for 1,3-propanediol production are placed under the transcriptional control of promoters selected from a group consisting of: rbcL, ntcA, nblA, isiA, petJ, petE, sigB, lrtA, htpG, hspA, clpB1, hliB, ggpS, psbA2, psaA, nirA, crhC, and srp. The promoters hspA, clpB1, and hliB can be induced by heat shock (raising the growth temperature of the host cell culture from 30° C. to 40° C.), cold shock (reducing the growth temperature of the cell culture from 30° C. to 20° C.), oxidative stress (for example by adding oxidants such as hydrogen peroxide to the culture), or osmotic stress (for example by increasing the salinity). The promoter sigB can be induced by stationary growth, heat shock, and osmotic stress. The promoters ntcA and nblA can be induced by decreasing the concentration of nitrogen in the growth medium and the promoters psaA and psbA2 can be induced by low light or high light conditions. The promoter htpG can be induced by osmotic stress and heat shock. The promoter crhC can be induced by cold shock. An increase in copper concentration can be used in order to induce the promoter petE, whereas the promoter petJ is induced by decreasing the copper concentration. The promoter sip can be induced by the addition of IPTG (isopropyl β-D-1-thiogalactopyranoside). Additional details of these promoters can be found, for example, in PCT/EP2009/060526, which is incorporated by reference herein in its entirety.

In an embodiment, the inducible promoters are selected from the group consisting of: PntcA, PnblA, PisiA, PpetJ, PpetE, PggpS, PpsbA2, PpsaA, PsigB, PlrtA, PhtpG, PnirA, PhspA, PclpB1, PhliB, PcrhC, PziaA, PsmtA, PcorT, PnrsB, PaztA, PbmtA, Pbxa1, PzntA, PczrB, PnmtA and Psrp.

In certain other preferred embodiments, truncated or partially truncated versions of these promoters including only a small portion of the native promoters upstream of the transcription start point, such as the region ranging from −35 to the transcription start can often be used. Furthermore, the introduction of nucleotide changes into the promoter sequence, e.g. into the TATA box, the operator sequence and/or the ribosomal binding site (RBS) can be used to tailor or optimize the promoter strength and/or its induction conditions, such as the concentration of inducer compound.

In an embodiment, the promoter used to regulate expression of 1,3-propanediol pathway genes is the Psrp promoter (SEQ ID NO: 1). In another embodiment, the promoter is PnblA₇₁₂₀ (the phycobilisome degradation protein promoter from Nostoc sp. PCC 7120 (SEQ ID NO: 2). In an embodiment, the promoter is PrbcL₆₈₀₃ (the constitutive ribulose 1,5-bisphosphate carboxylase/oxygenase large subunit promoter from Synechocystis sp. PCC 6803 (SEQ ID NO: 3). Another promoter that can be used is PsmtA₇₀₀₂ (the promoter for prokaryotic metallothionein-related protein from Synechococcus sp. PCC 7002; (SEQ ID NO: 4). The repressor/promoter system ziaR-PziaA₆₈₀₃ (the zinc-inducible promoter from Synechocystis sp. PCC 6803; (SEQ ID NO: 5) can also be used.

### Production of 1,3-Propanediol in Cyanobacteria

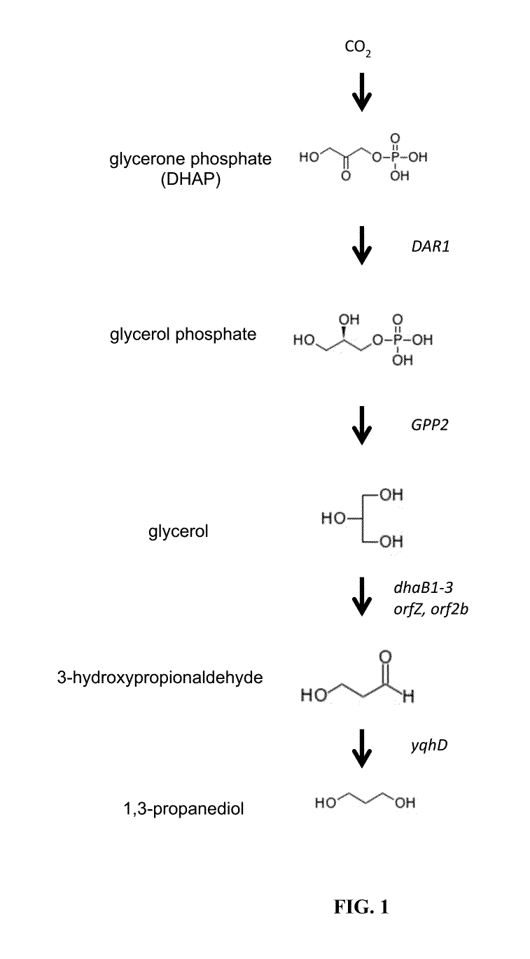

Cyanobacteria can be modified to produce 1,3-propanediol. A biosynthetic pathway for production of 1,3-propanediol in cyanobacteria is shown in FIG. 1. The substrate dihydroxyacetone phosphate (also called glycerone phosphate and abbreviated as DHAP) is already present in the cyanobacterial cell. Addition of genes encoding the enzymes involved in this pathway can result in the production of 1,3-propanediol.

In an embodiment, the biochemical pathway from CO₂ to 1,3-propanediol involves several steps. The substrates are:
CO₂→→→Dihydroxyacetone phosphate→glycerol phosphate→glycerol→3-hydroxypropionaldehyde→1,3-propanediol

To create the 1,3-propanediol biosynthetic pathway from CO₂ as the carbon source, the following genes can be inserted into the cyanobacterial cell:
DAR1-GPP2-dhaB1-3-(orfZ and orf2b)-yqhD

A demonstration of the construction of plasmids for the production of 1,3-propanediol is shown in Example 4. A listing of several plasmids that were constructed is shown in Table 4. An example of a successful transformation of the 1,3-propanediol constructs to cyanobacteria is shown in Example 5. Verification of the successful transformation is shown in Example 6. A suitable method for determining the level of 1,3-propanediol that is produced is shown in Example 8.

As mentioned in the background section, U.S. Pat. No. 8,216,816 describes a prophetic example of another method of biological engineering for the production of 1,3-propanediol in microorganisms. The prophetic method described in the U.S. Pat. No. 8,216,816 describes genes encoding the following enzymes: dar1, gpp2, dhaB1-3, and dhaT. However, there is no teaching of the presence of reactivases (such as orfZ and orf2b), which are needed for successful production of the product. Also, the U.S. Pat. No. 8,216,816 describes the use of the dhaT gene, which has a relatively low enzymatic activity (Nakamura et al., 2003, Curr. Opin. Biotech. 14:454-459).

In contrast, the method described herein differs in several ways. In an embodiment, genes encoding the reactivase enzymes orfZ and orf2b are present, which have been found to be required for successful production of product. Also, in an embodiment, rather than the dhaT enzyme mentioned in the U.S. Pat. No. 8,216,816, the enzyme yqhD is used to catalyze the conversion of 3-hydroxypropanal to 1,3-propanediol. The enzyme yqhD has a higher activity than dhaT (Nakamura et al., 2003, supra).

A biosynthetic pathway consisting of DAR1 (glycerol-3-phosphate dehydrogenase) and GPP2 (glycerol-3-phosphatase) is capable of converting glycerone phosphate (DHAP) to glycerol. 1,3-propanediol production can then be achieved with genes encoding a coenzyme B₁₂-dependent glycerol dehydratase (dhaB1-3), a coenzyme B₁₂ reactivase (orfZ and orf2b) and an alcohol dehydrogenase (yqhD), which is also termed “1,3-propanediol oxidoreductase.”

The terms “glycerol-3-phosphate dehydrogenase” and “DAR1” refer to an enzyme that is involved in glycerophospholipid metabolism and responses to cellular osmotic stress in yeast. The enzyme facilitates the production of glycerol phosphate from glycerone phosphate. A “DAR1 gene” refers to the gene encoding an enzyme that facilitates the production of glycerol phosphate from glycerone phosphate. In one embodiment of the invention, the DAR1 gene is derived from S. cerevisiae, nucleic acid accession # NM_{—}001180081 and protein accession # NP_{—}010262.1. In another embodiment, the invention provides a recombinant photosynthetic microorganism that includes at least one heterologous DNA sequence encoding at least one polypeptide that catalyzes a substrate to product conversion that leads to the synthesis of glycerol phosphate from glycerone phosphate. In an embodiment, the DAR1 enzyme is a member of the enzyme class EC#1.1.1.8. In an embodiment, the DAR1 nucleotide sequence is SEQ ID NO: 6, and the DAR1 amino acid sequence is SEQ ID NO: 7.

The terms “glycerol-3-phosphatase” and “GPP2” (also known as “YER062C” and “HOR2” refer to an enzyme that is required for glycerol biosynthesis in yeast. In yeast, the enzyme has been found to be involved in responses to various cellular stresses, such as osmotic and oxidative stress (Pahlman et al., J Biol Chem. 276:3555-3563; 2001). The enzyme can catalyze the formation of glycerol from glycerol phosphate. A “GPP2 gene” refers to the gene encoding an enzyme that facilitates the production of glycerol from glycerol phosphate. In an embodiment, GPP2 is encoded by nucleic acid accession # NM_{—}001178953.1 and protein accession # NP_{—}010984.1, derived from S. cerevisiae. In another embodiment, the invention provides a recombinant photosynthetic microorganism that includes at least one heterologous DNA sequence encoding at least one polypeptide that catalyzes a substrate to product conversion that leads to the synthesis of glycerol from glycerol phosphate. In an embodiment, the GPP2 enzyme is a member of the enzyme class EC#3.1.3.21. In an embodiment, the GPP2 nucleotide sequence is SEQ ID NO: 8, while the GPP2 amino acid sequence is SEQ ID NO: 9.

The terms “coenzyme B12-dependent glycerol dehydratase” refers to a group of three genes, collectively termed “dhaB1-3,” that encode an enzyme complex that is involved in glycerolipid metabolism, which is capable of catalyzing the formation of 3-hydroxypropionaldehyde from glycerol. The enzyme complex is comprised of three polypeptides. In an embodiment, an operon comprising all three dhaB (dhaB1, dhaB2, dhaB3) nucleotide sequences (SEQ ID NO: 10), is used. In an embodiment, dhaB1 has a nucleic acid sequence of SEQ ID NO: 11 and amino acid sequence of SEQ ID NO: 12; dhaB2 has a nucleic acid sequence of SEQ ID NO: 13 and amino acid SEQ ID NO: 14; and dhaB3 has a nucleic acid sequence of SEQ ID NO: 15 and amino acid SEQ ID NO: 16).

Together, the three polypeptides encoded by dhaB1-3 form an enzyme that facilitates the production of 3-hydroxypropionaldehyde from glycerol. In an embodiment, the gene sequence is nucleic acid accession # CP000647.1:3846008 . . . 3848700 and protein accession # ABR78884.1, ABR78883.1, and ABR78882.1, derived from Klebsiella pneumoniae subspecies pneumoniae (Shroeter) Trevisan (ATCC#700721, herein referred to as K. pneumoniae). In another embodiment, the invention provides a recombinant photosynthetic microorganism that includes at least one heterologous DNA sequence encoding at least one polypeptide that catalyzes a substrate to product conversion that leads to the synthesis of hydroxypropionaldehyde from glycerol. In an embodiment, the dhaB1-3 enzyme is a member of the enzyme class EC#4.2.1.30.

The terms “orfZ”, and “orf2b” refer to glycerol dehydratase reactivase enzymes. In an embodiment, the genes are derived from K. pneumoniae. In an embodiment, an artificially created operon (SEQ ID NO: 17) encoding both orfZ and orf2b is used. In an embodiment, the gene sequence of orfZ (SEQ ID NO: 18) is nucleic acid accession # CP000647.1:3844172 . . . 3845995 and the protein accession # is ABR78881.1 (“glycerol dehydratase activator”; SEQ ID NO: 19). This enzyme has chaperone-like activity and apparently functions to remove damaged coenzyme B₁₂ from glycerol dehydratase that has become inactivated. In an embodiment, the gene sequence of orf2b (“glycerol dehydratase reactivation factor small subunit”; SEQ ID NO: 20) is JF260927.1:6577 . . . 6930 and the protein sequence accession # is AEL12184.1 (SEQ ID NO: 21).

The term “yqhD” refers to a gene encoding an alcohol dehydrogenase that can function as a 1,3-propanediol oxidoreductase. The enzyme can catalyze the formation of 1,3-propanediol from 3-hydroxypropionaldehyde. In an embodiment, the gene is derived from E. coli. In an additional embodiment, the gene is nucleic acid accession # NC_{—}010473.1:3251122 . . . 3252285 and the protein accession is # YP_{—}001731875.1. In another embodiment, the invention provides a recombinant photosynthetic microorganism that includes at least one heterologous DNA sequence encoding at least one polypeptide that catalyzes a substrate to product conversion that leads to the synthesis of 1,3-propanediol from 3-hydroxypropionaldehyde. In an embodiment, the YqhD enzyme is a member of the enzyme class EC#1.1.1.202. In an embodiment, the yqhD nucleotide sequence is SEQ ID NO: 22, and the YqhD amino acid sequence is SEQ ID NO: 23.

### Glyerol Production in Cyanobacteria

A portion of the biosynthetic pathway for 1,3-propanediol production involves the production of glycerol, as shown below. The precursor glycerone phosphate is typically readily available in the cyanobacterial cell. By adding the two genes DAR1 and GPP2 to a cyanobacterial cell, glycerol can be produced, as shown in Examples 7 and 12.

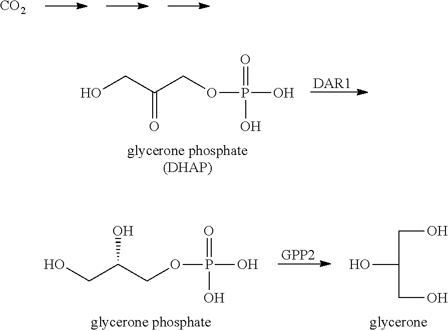

### Glyerol Feed to Cyanobacteria to Produce 1,3-Propanediol

Certain cyanobacterial species contain glycerol transporter proteins and can therefore take up glycerol from the medium. Glycerol is currently commonly available as a waste material from biodiesel production. Accordingly, in an embodiment, a cyanobacterial species having an endogenous glycerol transporter protein, further having at least some of the 1,3-propanediol pathway genes (dhaB1-3, orf2B/orfZ, and yqhD) described herein can take up exogenously added glycerol to produce the 1,3-propanediol product. The glycerol feed can be a one-time dose, or can be added intermittently, or can be added constantly. In an embodiment, the glycerol is added during the dark phase of a culture's light/dark cycle to promote glycerol uptake.

### Transformation of Cyanobacterial Cells

Cyanobacteria can be transformed by several suitable methods. Exemplary cyanobacteria that can be transformed with the nucleic acids described herein include, but are not limited to, Synechocystis, Synechococcus, Acaryochloris, Anabaena, Thermosynechococcus, Chamaesiphon, Chroococcus, Cyanobacterium, Cyanobium, Dactylococcopsis, Gloeobacter, Gloeocapsa, Gloeothece, Microcystis, Prochlorococcus, Prochloron, Chroococcidiopsis, Cyanocystis, Dermocarpella, Myxosarcina, Pleurocapsa, Stanieria, Xenococcus, Arthrospira, Borzia, Crinalium, Geitlerinema, Halospirulina, Leptolyngbya, Limnothrix, Lyngbya, Microcoleus, Cyanodictyon, Aphanocapsa, Oscillatoria, Planktothrix, Prochlorothrix, Pseudanabaena, Spirulina, Starria, Symploca, Trichodesmium, Tychonema, Anabaenopsis, Aphanizomenon, Calothrix, Cyanospira, Cylindrospermopsis, Cylindrospermum, Nodularia, Nostoc, Chlorogloeopsis, Fischerella, Geitleria, Nostochopsis, Iyengariella, Stigonema, Rivularia, Scytonema, Tolypothrix, Cyanothece, Phormidium, Adrianema, and the like.

Exemplary methods suitable for transformation of Cyanobacteria, include, as nonlimiting examples, natural DNA uptake (Chung, et al. (1998) FEMS Microbiol. Lett. 164: 353-361; Frigaard, et al. (2004) Methods Mol. Biol. 274: 325-40; Zang, et al. (2007) J. Microbiol. 45: 241-245), conjugation, transduction, glass bead transformation (Kindle, et al. (1989) J. Cell Biol. 109: 2589-601; Feng, et al. (2009) Mol. Biol. Rep. 36: 1433-9; U.S. Pat. No. 5,661,017), silicon carbide whisker transformation (Dunahay, et al. (1997) Methods Mol. Biol. (1997) 62: 503-9), biolistics (Dawson, et al. (1997) Curr. Microbiol. 35: 356-62; Hallmann, et al. (1997) Proc. Natl. Acad. USA 94: 7469-7474; Jakobiak, et al. (2004) Protist 155:381-93; Tan, et al. (2005) J. Microbiol. 43: 361-365; Steinbrenner, et al. (2006) Appl Environ. Microbiol. 72: 7477-7484; Kroth (2007) Methods Mol. Biol. 390: 257-267; U.S. Pat. No. 5,661,017) electroporation (Kjaerulff, et al. (1994) Photosynth. Res. 41: 277-283; Iwai, et al. (2004) Plant Cell Physiol. 45: 171-5; Ravindran, et al. (2006) J. Microbiol. Methods 66: 174-6; Sun, et al. (2006) Gene 377: 140-149; Wang, et al. (2007) Appl. Microbiol. Biotechnol. 76: 651-657; Chaurasia, et al. (2008) J. Microbiol. Methods 73: 133-141; Ludwig, et al. (2008) Appl. Microbiol. Biotechnol. 78: 729-35), laser-mediated transformation, or incubation with DNA in the presence of or after pre-treatment with any of poly(amidoamine) dendrimers (Pasupathy, et al. (2008) Biotechnol. J. 3: 1078-82), polyethylene glycol (Ohnuma, et al. (2008) Plant Cell Physiol. 49: 117-120), cationic lipids (Muradawa, et al. (2008) J. Biosci. Bioeng. 105: 77-80), dextran, calcium phosphate, or calcium chloride (Mendez-Alvarez, et al. (1994) J. Bacteriol. 176: 7395-7397), optionally after treatment of the cells with cell wall-degrading enzymes (Perrone, et al. (1998) Mol. Biol. Cell 9: 3351-3365); and biolistic methods (see, for example, Ramesh, et al. (2004) Methods Mol. Biol. 274: 355-307; Doestch, et al. (2001) Curr. Genet. 39: 49-60; all of which are incorporated herein by reference in their entireties).

### Culturing the Cyanobacterial Cells

In an embodiment, 1,3-propanediol is synthesized in cyanobacterial cultures by preparing host cyanobacterial cells having the gene constructs discussed herein, and growing cultures of the cells.

The choice of culture medium can depend on the cyanobacterial species. In an embodiment of the invention, the following BG-11 medium for growing cyanobacteria can be used (Table 1 and Table 2, below). When saltwater species are grown, Instant Ocean (35 g/L) and vitamin B₁₂ (1 μg/ml) can be added to the culture medium.

TABLE 1
Exemplary Culture Medium Composition

| | | |
|---|---|---|
| | Amount | Final |
| Compound | (per liter) | Concentration |

| NaNO₃ | 1.5 | g | 17.6 | mM |
|---|---|---|---|---|
| K₂HPO₄ | 0.04 | g | 0.23 | mM |
| MgSO₄•7H₂O | 0.75 | g | 3.04 | mM |
| CaCl₂•2H₂O | 0.036 | g | 0.24 | mM |
| Citric acid | 0.006 | g | 0.031 | mM |
| Ferric ammonium citrate | 0.006 | g | — | |
| EDTA (disodium salt) | 0.001 | g | 0.0030 | mM |
| NaCO₃ | 0.02 | g | 0.19 | mM |

| | | | |
|---|---|---|---|
| Trace metal mix A5 | 1.0 | ml | — |

TABLE 2
Trace Metal Mix

| | | | Concentration in | |
|---|---|---|---|---|
| Trace Metal mix A5 | | | Final Medium | |
| H₃BO₃ | 2.86 | g | 46.26 | μM |
| MnCl₂•4H₂O | 1.81 | g | 9.15 | μM |
| ZnSO₄•7H₂O | 0.222 | g | 0.772 | μM |
| NaMoO₄•2H₂O | 0.39 | g | 1.61 | μM |
| CuSO₄•5H₂O | 0.079 | g | 0.32 | μM |
| Co(NO₃)₂•6H₂O | 49.4 | mg | 0.170 | μM |

| | | | |
|---|---|---|---|
| Distilled water | 1.0 | L | — |

In an embodiment, the cells are grown autotrophically, and the only carbon source is CO₂. In another embodiment, the cells are grown mixotrophically, for example with the addition of a carbon source such as glycerol.

The cultures can be grown indoors or outdoors. The cultures can be axenic or non-axenic. In another embodiment, the cultures are grown indoors, with continuous light, in a sterile environment. In another embodiment, the cultures are grown outdoors in an open pond type of photobioreactor.

In an embodiment, the cyanobacteria are grown in enclosed bioreactors in quantities of at least about 100 liters, 500 liters, 1000 liters, 2000 liters, 5,000 liters, or more. In an embodiment, the cyanobacterial cell cultures are grown in disposable, flexible, tubular photobioreactors made of a clear plastic material.

The light cycle can be set as desired, for example: continuous light, or 16 hours on and 8 hours off, or 14 hours on and 10 hours off, or 12 hours on and 12 hours off.

Isolation and Purification of 1,3-Propanediol from the Cyanobacterial Cultures

Various methods can be used to remove the 1,3-propanediol from the cyanobacterial culture medium. For a review of several currently used methods to separate and purify 1,3-propanediol, for example, see Xiu et al., Appl. Microbiol. Biotechnol. 78:917-926; 2008.

In an embodiment, the propanediol is separated from the culture medium periodically as the culture is growing. For example, the culture medium can be separated from the cells, followed by a filtration step. The propanediol can then be removed from the filtrate. The culture medium can be recycled back into the culture, if desired, or new culture medium can be added. In another embodiment, the propanediol is removed from the culture at the end of the batch run.

A method for isolating 1,3-propanediol from the fermentation broth of a genetically modified E. coli culture is described in U.S. Pat. No. 7,919,658 to Adkesson et al. The method involves filtering the particulates out of the culture broth, running the broth through an ion exchange column, and then distilling the resulting liquid to produce substantially purified 1,3-propanediol.

Another method of separating polyol products from the culture producing it is described in International Patent Application No. WO/2000/024918 to Fisher et al. This application describes a pre-treatment step that can be used to separate the cells from the polyol-containing solution without killing the cell culture. Additional steps can include flotation or flocculation to remove proteinaceous materials, followed by ion exchange chromatography, activated carbon treatment, evaporative concentration, precipitation and crystallization.

A process for reclaiming 1,3-propanediol from operative fluids such as antifreeze solutions, heat transfer fluids, deicers, lubricants, hydraulic fluids, quenchants, solvents and absorbents, is disclosed in U.S. Pat. No. 5,194,159 to George et al. The method involves contacting the fluid with semi-permeable membranes under reverse osmosis.

U.S. Pat. No. 5,510,036 to Woyciesjes et al. discloses a process for the purification and removal of contaminants (such as heavy metals oils and organic contaminants) in a polyol-containing solution, wherein the process involves lowering the pH and adding precipitating, flocculating, or coagulating agents, which can be followed by filtration and an ion exchange chromatography step.

The present invention is further described by the following non-limiting examples. However, it will be appreciated that those skilled in the art, on consideration of this disclosure, may make modifications and improvements within the spirit and scope of the present invention.

### EXAMPLES

### Example 1

### General Methods

Restriction endonucleases were purchased from New England Biolabs (New England Biolabs (NEB), Ipswich, Mass.), unless otherwise noted. PCR was performed using an Eppendorf Mastercycler thermocycler (Eppendorf, Hauppauge, N.Y.), using Phire II Hot Start polymerase or Taq DNA polymerase (NEB) for diagnostic amplifications, and Phusion polymerase or Crimson LongAmp Taq Polymerase (NEB) for high fidelity amplifications. PCR temperature profiles were set up as recommended by the polymerase manufacturer. Cloning was performed in E. coli using XL10-Gold Ultracompetent cells (Agilent Technologies, Santa Clara, Calif.) following the manufacturer's protocol. TOPO cloning kits (Zero Blunt TOPO PCR Cloning kit) were purchased from Invitrogen (Invitrogen, Carlsbad, Calif.), and were used according to the manufacturer's protocol.

BG-11 stock solution was purchased from Sigma Aldrich (Sigma Aldrich, St. Louis, Mo.). Marine BG-11 (MBG-11) was prepared by dissolving 35 g Instant Ocean (United Pet Group, Inc, Cincinnati, Ohio) in 1 L water and supplementing with BG-11 stock solution. Vitamin B₁₂ (Sigma Aldrich) was supplemented to MBG-11 to achieve a final concentration of 1 μg/L, as needed. Solid media (agar plates) were prepared similarly to liquid media, with the addition of 1% (w/v) phyto agar (Research Products International Corp, Mt. Prospect, Ill.). Stock solutions of the antibiotics spectinomycin (100 mg/ml) and kanamycin (50 mg/ml) were purchased from Teknova (Teknova, Hollister, Calif.). Stock solution of the antibiotic gentamycin (10 mg/ml) was purchased from MP Biomedicals (MP Biomedicals, Solon, Ohio).

### Example 2

### SLIC Method (Sequence- and Ligation-Independent Cloning)

Primers were designed with 5′ sequences that overlapped the target vector at the desired restriction site, or which overlapped the next PCR product if inserting more than one product at a time. The overlapping sequence was typically 30 base pairs (bp) long. PCR products were amplified from genomic DNA (Klebsiella or Saccharomyces) or from whole cells (E. coli) and gel-purified. Target vectors were digested with appropriate restriction enzymes and gel-purified. To generate the 30-bp sticky ends, digested target vector (200 ng-1 μg) and each PCR product (20 ng-1 μg) were treated with 0.5 U of T4 DNA polymerase from NEB in NEB buffer 2 plus BSA (with no dNTP's) and incubated at room temperature for 15 minutes per 10 bp overlap (45 minutes for a 30 bp overlap). Reactions were stopped by adding 1/10 volume of 10 mM dCTP (or other single dNTP). Equimolar amounts (1:1 or 1:1:1, etc.) of T4-treated vector and insert(s) were combined in 8 μl volume in a PCR tube. 10×T4 ligase buffer, 1 μl, was added to the tube. Using a thermal cycler, reactions were heated to 65° C. for 10 minutes, then slowly ramped down to 37° C. (10% ramp speed). RecA protein from NEB, 20 ng in 1 ml 10× RecA buffer, was added to the tube, which was incubated at 37° C. for 30 minutes. 5 μl of the reaction was used for E. coli transformation.

### Example 3

### Preparation of the RSF1010-Derived Plasmid Backbone for the Expression Vectors

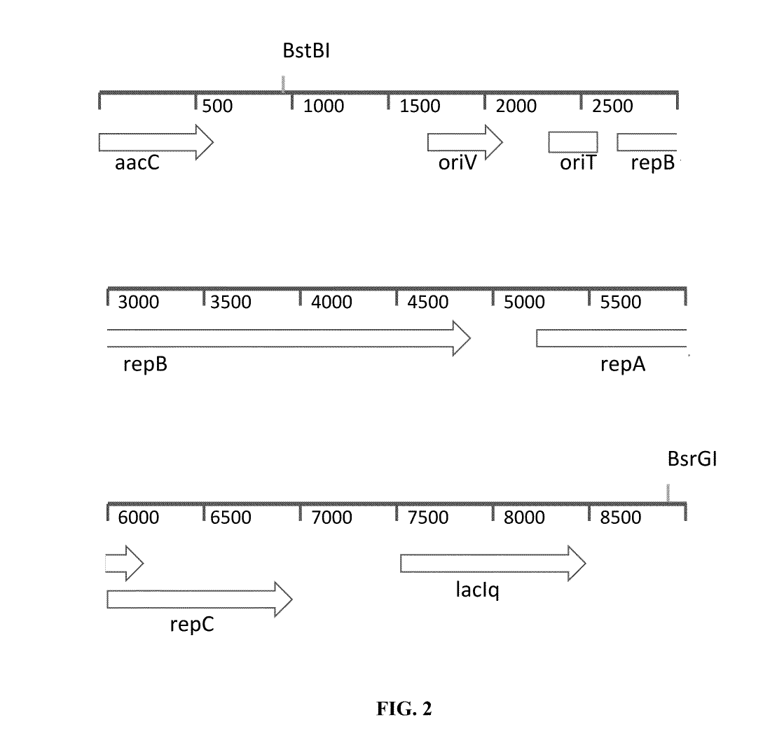

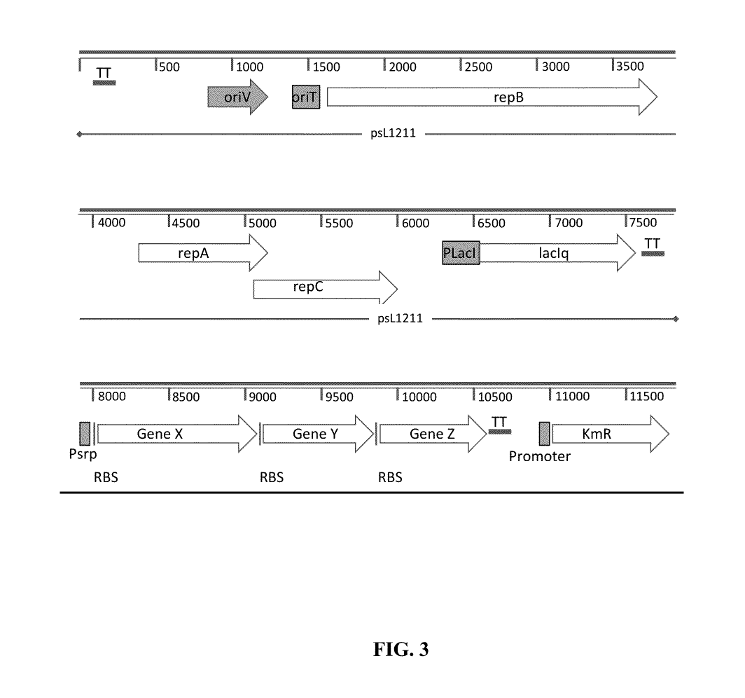

Broad-host range plasmids described herein are based off of the RSF1010-derivative plasmid pSL1211, as shown in FIG. 2. An IPTG-inducible srp promoter and a kanamycin resistance gene were ligated into pSL1211, generating the plasmid pABb, to be used as a backbone plasmid for the heterologous expression of propanediol genes (FIG. 3).

### Example 4

### Construction of Plasmids for 1,3-Propanediol Production in Cyanobacteria

A biosynthetic pathway for the production of 1,3-propanediol in cyanobacteria was constructed utilizing the steps shown in FIG. 1. The recombinant 1,3-propanediol-producing genes were designed to have polycistronic expression driven by a single promoter in a single operon with the genes arranged in the same order as they are in the pathway.

Each gene was designed to have its own RBS (ribosome-binding site). The genes were inserted into the RSF1010-derived plasmid backbone. The RSF1010 origin of replication served as a replication origin for both E. coli and for the cyanobacterial strains. The primers used for the plasmid construction are shown below in Table 3.

TABLE 3
Primers for Construction of 1,3-Propanediol Producing Plasmids

| Primer | |
|---|---|
| Name | Primer Sequence |
| DAR1 | gtcaatcccatatgtagatctcctGAATTCctaatcttcatgtagatctaattctt (SEQ ID NO: 24) |
| R1 | |
| DAR1 | aggagtctgttatgaacggtaccatgAATTcatgtctgctgctgctgataga (SEQ ID NO: 25) |
| Fn | |
| DAR1 | Atgtttatggaggactgacctagatgaattcatgtctgctgctgctgataga (SEQ ID NO: 26) |
| Fr | |
| GPP2 | atgaagattagGAATTCaggagatctacatatgggattgactactaaacctct (SEQ ID NO: 27) |
| F1 | |
| GPP2 | gatcttttcatCCTGCAGGctcctGAATTCttaccatttcaacagatcgtcct (SEQ ID NO: 28) |
| R1 | |
| dha F1 | tgaaatggtaaGAATTCaggagCCTGCAGGatgaaaagatcaaaacgatttgc (SEQ ID NO: 29) |
| dha F2 | aatgtgtggatcagcaggacgcactgaccgGAATTCaggagCCTGCAGGatgaaaagatcaaaacgatttg |
| | c (SEQ ID NO: 30) |
| dha R1 | gttcatcGCTAGCtctcctcttGGCGCGCCttaattcgcctgaccggcc (SEQ ID NO: 31) |
| dhaB3_ | Gcaggcggagctgctggcg (SEQ ID NO: 32) |
| R | |
| yqhD Fl | aattaaGGCGCGCCaagaggagaGCTAGCgatgaacaactttaatctgcacacc (SEQ ID NO: 33) |
| yqhD | cgctactgccgccaggcaaattctgtttccTGCAGGCGCGCCgcttagcgggcggcttcg (SEQ ID NO: |
| R1 | 34) |
| yqhD Rr | CTAGAGCATGCAGATCTAGCGGCCGCTCGATGCAGGCGCGCCgcttagcgggcg |
| | gcttcg (SEQ ID NO: 35) |
| yqhD_L2 | ACTGTTCCACGGTGTGTACAAAGG (SEQ ID NO: 36) |
| orf2b | gtcaggcgaattaaGGCGCGCCaggagaactagtaatgtcgctttcaccgccagg (SEQ ID NO: 37) |
| Fasc | |
| orf2b | GCTAGCtctcctcttGGCGCGCCtcagtttctctcacttaacggc (SEQ ID NO: 38) |
| Rasc | |

The genes DAR1 (SEQ ID NO: 6) and GPP2 (SEQ ID NO: 8) were amplified from wild type Saccharomyces cerevisiae using primers DAR1 F1, DAR1 R1, GPP2 F1, and GPP2 R1 in standard PCR reactions. Overlap PCR was used to combine DAR1 and GPP2 into a single PCR product. This was ligated into a TOPO blunt cloning vector per the manufacturer's instructions, resulting in pAB1002. DAR1 and GPP2 PCR products were cloned into plasmid pABb digested with EcoRI and SbfI in a standard SLIC reaction, resulting in pAB1001 (SEQ ID NO: 39).

The nucleic acid sequences dhaB1-3 (SEQ ID NO: 10) and orfZ (SEQ ID NO: 17) were amplified from wild type K. pneumoniae genomic DNA as a single PCR product using primers dha F2 and dha R1 in standard PCR reactions. The yqhD gene was amplified from wild type E. coli using primers yqhD F1 and yqhD R1 in standard PCR reactions. The PCR products containing the dhaB1-3-orfZ-yqhD genes were cloned into vector pABb digested with restriction enzymes EcoRI and SbfI in a standard SLIC reaction, resulting in plasmid pAB1003 (SEQ ID NO: 40). Primers dha F1 and yqhD R1 were used to amplify dhaB1-3-orfZ-yqhD from pAB1003. This was ligated into a TOPO blunt cloning vector according to the manufacturer's instructions, resulting in pAB1005.

Plasmid pAB1002 was digested with SbfI and SpeI and the 5.5-kb fragment was gel-purified and treated as the vector. Plasmid pAB1005 was digested with NsiI and SpeI and the 5.9-kb fragment was gel-purified and treated as the insert. The digested fragments were ligated together, resulting in pAB1014. The orf2b gene was amplified from wild type K. pneumoniae genomic DNA as a single PCR product using primers orf2b Fasc and orf2b Rasc. The product was gel-purified and recombined using GENEART Seamless Cloning and Assembly Kit from Invitrogen into pAB1014 which had been digested with AscI, resulting in pAB1035.

DAR1-GPP2 was amplified from pAB1014 using primers DAR1 Fn and GPP2 R1; dhaB1-3-orfZ-yqhD was amplified from pAB1014 using primers dha F1 and yqhD Rr; the PCR products were recombined into pAB412 digested with EcoRI and XhoI using the GENEART Seamless Cloning and Assembly Kit, resulting in pAB1034. pAB1034 was digested with AsiSI and BsrGI. The orf2b with portions of orfZ and yqhD was PCR-amplified from pAB1035 using primers dhaB3_R and yqhD_L2. The PCR product was recombined into pAB1034 AsiSI/BsrGI, resulting in pAB1040 (SEQ ID NO: 41). DAR1-GPP2-dhaB1-3-orfZ-orf2b-yqhD was PCR-amplified from pAB1040 using primers DAR1 Fr and yqhD Rr and recombined into pAB415 digested with EcoRI and XhoI, resulting in pAB1050.

The sequences of pAB1040 and pAB1050 were confirmed using both digestion with the restriction enzyme AflII and by sequencing. The plasmid pAB1070 contained the above-described 1,3-propanediol pathway genes controlled by the zinc-inducible promoter ziaR-PziaA₆₈₀₃.

Several combinations of constructs using different promoters and different plasmids were prepared as shown in Table 4, below.

TABLE 4
1,3-Propanediol Plasmids

| | | | E coli | Cyanobac- |
|---|---|---|---|---|
| | | | Origin of | terial |
| Plasmid | | | Repli- | Origin of |
| Name | Promoter | Gene Cassette | cation | Replication |
| pAB1003 | Psrp | dhaB1-3-orfZ-yqhD | RSF1010 | RSF1010 |
| pAB1005 | Plac | dhaB1-3-orfZ-yqhD | pBR | N/A |
| pAB1014 | Plac | DAR1-GPP2-dhaB1- | pBR | N/A |
| | | 3-orfZ-yqhD | | |
| pAB1034 | PnblA₇₁₂₀ | DAR1-GPP2-dhaB1- | RSF1010 | RSF1010 |
| | | 3-orfZ-yqhD | | |
| pAB1035 | Plac | DAR1-GPP2-dhaB1- | pBR | N/A |
| | | 3-orfZ-orf2b-yqhD | | |
| pAB1040 | PnblA₇₁₂₀ | DAR1-GPP2-dhaB1- | RSF1010 | RSF1010 |
| | | 3-orfZ-orf2b-yqhD | | |
| pAB1050 | PrbcL₆₈₀₃ | DAR1-GPP2-dhaB1- | RSF1010 | RSF1010 |
| | | 3-orfZ-orf2b-yqhD | | |
| pAB1070 | ziaR- | DAR1-GPP2-dhaB1- | RSF1010 | RSF1010 |
| | PziaA | 3-orfZ-orf2b-yqhD | | |

### Example 5

### Transformation of Cyanobacterial Strains Synechococcus Sp. PCC 7002 and Synechocystis Sp. PCC 6803 with the 1,3-Propanediol Constructs

To confirm that the 1,3-propanediol genes are functional when transformed to cyanobacteria, cyanobacterial strains Synechococcus PCC 7002 and Synechocystis PCC 6803 were transformed with plasmids harboring various segments of the 1,3-propanediol pathway.

The transformation procedures were performed via conjugation, as follows: One week before the day of conjugation, cyanobacterial cells (e.g. PCC 7002 and PCC 6803) were inoculated with a fresh culture using a ˜1:10 dilution of an older (1 week) culture. E. coli cultures containing the plasmid(s) of interest and the helper plasmid pRL443 were started the night before the planned conjugation in ˜3 ml LB supplemented with the appropriate antibiotic(s). Four hours prior to conjugation, 30 ml of fresh LB medium (with appropriate antibiotic(s)) was inoculated with ˜0.5 ml of the overnight culture. The E. coli and cyanobacterial cultures were transferred to a 50 ml conical tube and centrifuged at 2,500×g for 10 minutes at room temperature to pellet the cells. The supernatant was decanted, and the cell pellets were resuspended in 1 ml LB (for the E. coli cultures) or (M)BG-11 (for cyanobacteria). The cells were then transferred to a microcentrifuge tube and centrifuged at 2,500×g for 10 minutes at room temperature. The decanting, resuspension, and centrifuge steps were repeated, resuspending each pellet in 300 μl LB or (M)BG-11, as appropriate. The cell resuspensions were diluted and the cells were counted. Approximately 3.6×10⁸ cells each of cyanobacteria, E. coli with plasmid pRL443, and E. coli with the plasmid of interest (aiming for about a 1:1:1 cell ratio), was placed in a microcentrifuge tube. The cell mixture was then centrifuged at 2,500×g for 5 minutes at room temperature. The supernatant was decanted and the pellet was resuspended in 950 μl (M)BG-11 and 50 μl LB. Sterilized cellulose nitrate membrane filters (Whatman) were transferred to (M)BG-11 (vitamin B₁₂)+5% LB agar plates. A 200 μl aliquot of the mixture was spread evenly on the filter. The agar plate was then placed in low light for two days. The filter was then transferred onto a fresh (M)BG-11 (+vitamin B₁₂) agar plate containing the appropriate selective antibiotic. MBG-11+vitamin B₁₂ plates had the following final antibiotic concentrations: spectinomycin, 100 μg/ml; kanamycin, 40 μg/ml. BG-11 plates had the following final antibiotic concentrations: spectinomycin, 15 μg/ml; kanamycin, 10 μg/ml. After 8-12 days, the presence of single colonies on the filters was monitored. Once single colonies were observed, the colonies were streaked onto a fresh selective plate (1st pass plate). The process was repeated (2nd pass plate). Once colonies were observed on the 2nd pass plate, the patch was taken and streaked onto an LB plate to check for potential E. coli contamination. Clean patches were used to perform colony PCR to test for the plasmid of interest.

### Example 6

### Colony PCR to Verify Transformation and Presence of the 1,3-Propanediol Pathway Genes

To confirm the presence of the 1,3-propanediol genes in the transformed cyanobacterial cells, streaks from colonies were resuspended in TE buffer and cells were disrupted with glass beads. Supernatants were used as a DNA template for PCR amplifications of fragments of the 1,3-propanediol pathway genes. The results of the PCR analysis confirmed the presence of the 1,3-propanediol genes in the host cells.

Cells from verified streaks were then used to inoculate 3 ml liquid BG-11 or MBG-11 vB₁₂ cultures supplemented with the appropriate antibiotics (MBG-11+vitamin B₁₂ medium had the following final antibiotic concentrations: spectinomycin, 100 μg/ml; kanamycin, 40 μg/ml; BG-11 medium had the following final antibiotic concentrations: spectinomycin, 15 μg/ml; kanamycin, 10 μg/ml) and incubated under a light intensity of 10-20 μmol photons m⁻²s⁻¹ at 37° C.

### Example 7

### Confirmation of Function of Initial Portion of 1,3-Propanediol Pathway in Cyanobacteria: Glycerone Phosphate to Glyerol Production

Several plasmid constructs having genes corresponding to the initial portion of the 1,3-propanediol pathway were prepared, as shown in Table 5, below. Synechocystis strain PCC 6803 was transformed with plasmid pAB1001 (SEQ ID NO: 39), following the method described in Example 5. The plasmid contained the DAR1 and GPP2 portion of the 1,3-propanediol biosynthetic pathway, in order to confirm that the first portion of the biosynthetic pathway (glycerone phosphate to glycerol) is functional in cyanobacteria.

The transformed cells were cultured in 100 ml of BG-11 in a 250 ml vented flask at 30° C. under a 12 hr/12 hr light dark cycle. One milliliter samples were taken periodically over a time period of one month. Each sample was processed by centrifuging the 1 ml culture at 12,000 rpm for two minutes and passing the supernatant through a 0.2 μm microcentrifuge column filter (SpinX). The filtered supernatant was analyzed on a Dionex instrument. Glycerol was measured using ion chromatography with pulsed amperometric detection. An IonPac ICE-AS 1 column (2 mm×250 mm) heated to 30° C. was used on a Dionex ICS-3000 IC system equipped with a disposable platinum electrode. The method was run using isocratic elution with 100 mM methanesulfonic acid at a flow rate of 0.2 mL/min for 30 minutes.

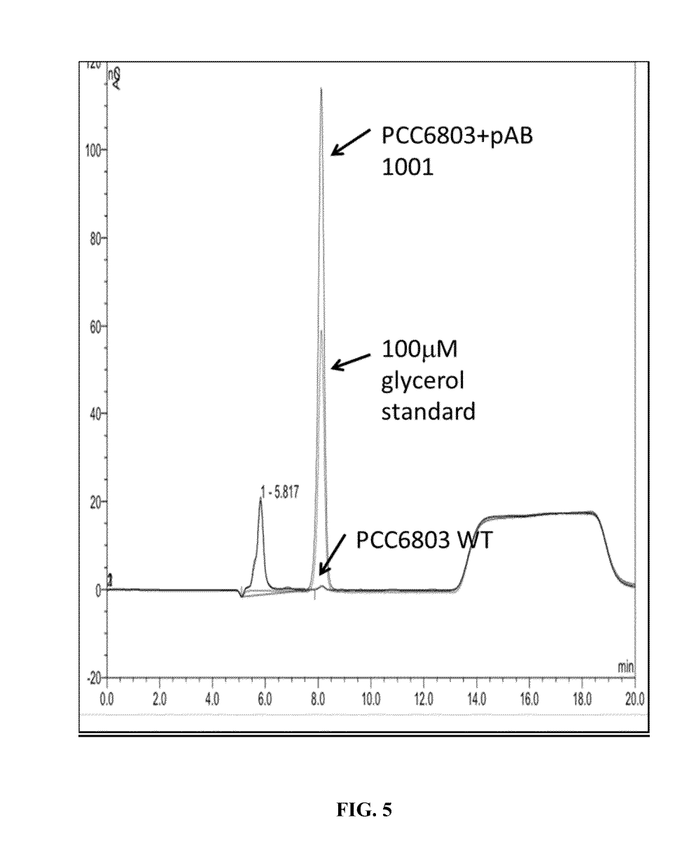

The results confirmed that glycerol was indeed produced in the transformed cyanobacteria (FIG. 5). The identity of the glycerol peak was confirmed by comparison with a pure glycerol standard, as shown in the figure. Glycerol was secreted into the surrounding medium with accumulated levels up to about 3 g/L after 30 days, at an average rate of ˜100 mg/L/day.

TABLE 5
Plasmid Constructs Having Glycerol-Producing Genes

| | | | E. coli | Cyanobac- |
|---|---|---|---|---|
| | | | Origin of | terial |
| Plasmid | | | Repli- | Origin of |
| Name | Promoter | Gene Cassette | cation | Replication |
| pAB1001 | Psrp | DAR1-GPP2 | RSF1010 | RSF1010 |
| pAB1002 | Plac | DAR1-GPP2 | pBR | N/A |
| pAB1028 | PrbcL₆₈₀₃ | DAR1-GPP2 | RSF1010 | RSF1010 |
| pAB1029 | PnblA₇₁₂₀ | DAR1-GPP2 | RSF1010 | RSF1010 |

### Example 8

### Confirmation of Production of 1,3-Propanediol from Glycerol in Cyanobacteria

To verify that the second part of the 1,3-propanediol pathway is functional in cyanobacteria, Synechococcus PCC 7002 was transformed with plasmid pAB1003 (SEQ ID NO: 40), which contains the last two steps encoding the enzymes in the biosynthetic pathway from glycerol to 1,3-propanediol (dhaB1-3-orfZ-yqhD). The cells were cultured in 25 ml of MBG-11, incubated at 37° C. under a 12 hr/12 hr light/dark cycle, shaking at 120 rpm. The cells were fed with a single one time feed of 1-2% glycerol. After 5-7 days, when cells were growing exponentially, the cultures were sampled to confirm the production of 1,3-propanediol.

A methanol/phosphate extraction was used to separate 1,3-propanediol produced from the culture. Five ml of cyanobacterial culture was saturated with dipotassium phosphate (˜6 g). This mixture was amended with methanol to a final methanol concentration of 30%, and was then vigorously shaken three times with five minute rest intervals. This extraction was incubated overnight at room temperature to allow phase separation. The upper methanol layer was collected, avoiding the interface, and evaporated to ˜100 μl (15× concentration) in a benchtop centrifugal evaporator. This extract was passed through a 0.2 μm filter prior to analysis.

The methanol extract was loaded onto a GC/MS using a liquid injection. 1,3-propanediol was measured using gas chromatography with flame ionization detection. A Stabilwax column (30 m length, 0.53 mm diameter, 1 μm film) was used on an Agilent 7890A GC system equipped with a 7683B liquid injector. A cyclo-uniliner was installed on the split/splitless injector and heated to 225° C. Two microliters were injected using a pulsed splitless program at 10 psi for 0.1 min. Using helium as the carrier gas at 50 cm/sec, separation was performed by running a linear thermal program from 80° C. to 200° C. at 24° C./min with a 5 minute hold at 200° C. Using this method, the retention time of 1,3-propanediol was 5.88 minutes.

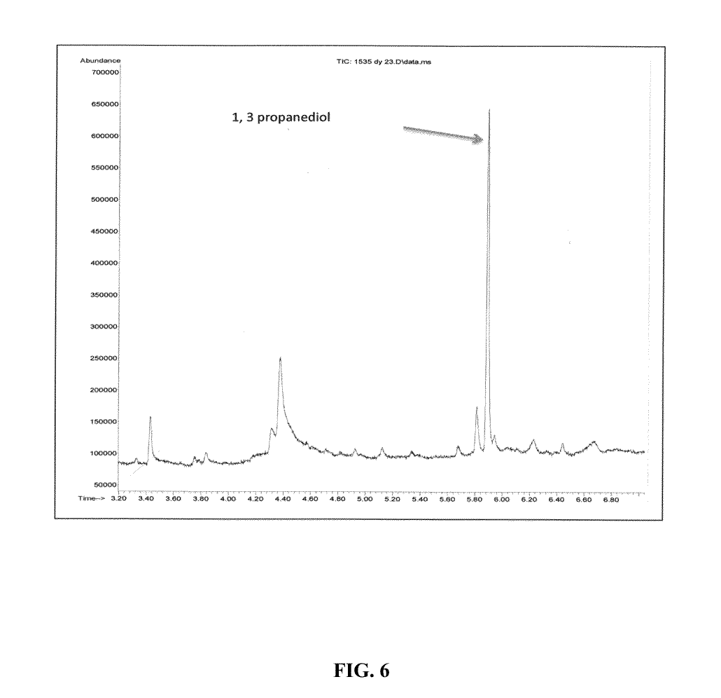

The results verified that 1,3-propanediol was produced in the transformed cyanobacteria when given a glycerol input feed: the cyanobacterial strain Synechococcus PCC 7002 transformed with the plasmid pAB1003 and fed with glycerol (1-2%) produced approximately 10 μM or approximately 1 mg/L 1,3-propanediol after one week of incubation (FIG. 6). The results verified that 1,3-propanediol was produced in the transformed cyanobacteria.

### Example 9

### Transformation of Cyanobacterial Strains Synechococcus Sp. PCC 7002 and Synechocystis Sp. PCC 6803 with Constructs Containing the Complete 1,3-Propanediol Pathway

To confirm that the complete biosynthetic pathway from glycerone phosphate to 1,3-propanediol can be successfully transformed to cyanobacteria to produce the 1,3-propanediol product, cyanobacterial strains Synechococcus PCC 7002, Synechocystis PCC 6803, and Anabaena are transformed with plasmids harboring the entire 1,3-propanediol pathway (DAR1+GPP2+dhaB1-3+orf2B/orfZ+yqhD).

In Synechococcus strain PCC 7002 the genes responsible for glycerol metabolism (e.g. glycerol kinase and/or glycerol dehydrogenase) are deleted to allow glycerol to only go towards 1,3-propanediol production. The first two genes, DAR1 and GPP2 are inserted onto a high copy plasmid to allow for higher expression of the glycerol production genes, to increase glycerol production. The genes dhaB1-3-orfZ-orf2b-yqhD remain on an RSF1010-based plasmid, as this was sufficient to product 1,3-propanediol from glycerol, as demonstrated in Example 8. In Synechocysis strain PCC 6803 the design is different than that suggested for PCC 7002, since the glycerol metabolic pathway does not exist in PCC 6803. The DAR1-GPP2 gene cassette remains under the control of a lower strength promoter. The genes dhaB1-3-orfZ-orf2b-yqhD gene cassette are under the control of a stronger promoter in hopes to limit glycerol accumulation and secretion. These separate gene cassettes remain on one plasmid, or can be placed onto separate plasmids.

The transformed cyanobacterial cells are tested to confirm that the transformation is successful. The cells are then grown for 2 weeks in a culture flask containing BG-11 medium, and a 16/8 light/dark cycle. 1,3-propanediol is extracted from the culture medium and quantified following the method described in Example 8. The results verify that 1,3-propanediol is produced in the transformed cyanobacteria. Thus, by use of this method, 1,3-propanediol can be produced in cyanobacterial cultures.

### Example 10

### Tolerance Testing to Determine Suitable Cyanobacterial Host Strain for 1,3-Propanediol Production

The tolerance of cyanobacterial strains Synechocystis sp. PCC 6803 and Synechococcus sp. PCC 7002 to the presence of accumulated 1,3-propanediol in the culture medium was examined by adding a one time bolus of varying amounts of 1,3-propanediol (ranging from 0.05% to 5%) to exponential phase cultures and comparing the growth of these cultures to a wild type culture with no addition. Growth was monitored by optical density (OD₇₅₀) for one week. There was no effect on the growth of Synechocystis sp. PCC 6803 in the presence of up to 1% 1,3-propanediol compared to the control (no addition of 1,3-propanediol). At 2% and 3% there was an inhibition effect resulting in a yellowing discoloration of the culture, slower growth and clumping. At 5% 1,3-propanediol, Synechocystis sp. PCC 6803 could not survive and bleached out after 3 days. There was no effect on the growth of Synechococcus sp. PCC 7002 with up to 1% 1,3-propanediol addition. However, a 2% addition was lethal resulting in a completely bleached culture.

### Example 11

### Scale-Up Production of the Genetically Enhanced Cyanobacteria in 200 Liter Photobioreactors and Collection of 1,3-Propanediol Product

A 10 L culture of Synechocystis PCC 6803 or Synechococcus PCC 7002 cells modified to contain a 1,3-propanediol gene cassette is inoculated into a final volume of 200 L in an indoor, temperature controlled photobioreactor with a 16 on/8 off light cycle, and grown for 2 months. At the end of the 2 month growth period, the spent culture medium is separated from the cellular material using filtration and flocculation. The cellular material is saved for other purposes. The culture medium is microfiltered and treated with a batch-wise ion exchange resin generally following the methods described in U.S. Pat. No. 7,919,658. The resulting 1,3-propanediol is further purified using methods known in the art.

Every 2 weeks, 50% of the culture medium is separated from the remaining cells and removed from the culture, and fresh replacement medium is added to the photobioreactor. The spent culture medium is filtered, pH treated, flocculated, filtered once again, then the resulting liquid is treated with a distillation procedure to result in substantially purified 1,3-propanediol.

### Example 12

### Scale-Up Production of Glyerol in Cyanobacteria

The first portion of the biosynthetic pathway from CO₂ to 1,3-propanediol, as described in Example 7, can also be utilized to produce glycerol in cyanobacteria, if desired. This involves the insertion of the DAR1 and GPP2 gene portion of the pathway to a suitable cyanobacterial strain. In a typical example, plasmid pAB1001 (SEQ ID NO: 39), containing the DAR1 and GPP2 genes, is transformed into Synechocystis PCC 6803 following the methods described in Example 5. The successful transformation is confirmed, and the cells are scaled-up to a large outdoor culture. Glycerol is collected from the culture medium. Identification of the glycerol peak is confirmed by retention time matching of a pure glycerol standard. By use of this method, glycerol can be produced in cyanobacteria.

Although the present invention has been described in considerable detail with reference to certain embodiments thereof, other embodiments are possible. Therefore, the spirit and scope of the appended claims should not be limited to the description of the embodiments contained therein.

What is claimed is:

1. A genetically enhanced cyanobacterial cell, comprising:
a) at least one promoter capable of regulating gene expression in cyanobacteria; and
b) a DAR1 gene, a GPP2 gene, the dhaB1-3 genes, an orfZ gene, an orf2b gene, and a yqhD gene,
wherein said genes are transcriptionally controlled by the at least one promoter, and further wherein said cell produces 1,3-propanediol.

2. The cyanobacterial cell of claim 1, wherein at least one of said genes is present in a location selected from the group consisting of an exogenously derived extrachromosomal plasmid, an endogenous plasmid-derived extrachromosomal plasmid, and on the cyanobacterial chromosome.

3. The cyanobacterial cell of claim 1, wherein said at least one promoter is selected from the group consisting of: Psrp, PnblA₇₁₂₀, PrbcL₆₈₀₃, PsmtA₇₀₀₂, and ziaR-PziaA₆₈₀₃.

4. The cyanobacterial cell of claim 1, wherein the DAR1 gene has at least 98% identity to SEQ ID NO: 6.

5. The cyanobacterial cell of claim 1, wherein the DAR1 gene encodes a polypeptide having at least 98% identity to SEQ ID NO: 7.

6. The cyanobacterial cell of claim 1, wherein the GPP2 gene has at least 98% identity to SEQ ID NO: 8.

7. The cyanobacterial cell of claim 1, wherein the GPP2 gene encodes a polypeptide having at least 98% identity to SEQ ID NO: 9.

8. The cyanobacterial cell of claim 1, wherein the dhaB1-3 genes have at least 98% identity to SEQ ID NO: 10.

9. The cyanobacterial cell of claim 1, wherein the dhaB1-3 genes encode three separate polypeptides, dhaB1, dhaB2, and dhaB3, wherein:
the DhaB1 polypeptide has at least 98% identity to SEQ ID NO: 12;
the DhaB2 polypeptide has at least 98% identity to SEQ ID NO: 14; and
the DhaB3 polypeptide has at least 98% identity to SEQ ID NO: 16.

10. The cyanobacterial cell of claim 1, wherein the orfZ and orf2b nucleic acid sequence has at least 98% identity to SEQ ID NO: 17.

11. The cyanobacterial cell of claim 1, wherein the orfZ gene encodes a polypeptide having at least 98% identity to SEQ ID NO: 19, and wherein the orf2b gene encodes a polypeptide having at least 98% identity to SEQ ID NO: 21.

12. The cyanobacterial cell of claim 1, wherein the yqhD gene has at least 98% identity to SEQ ID NO: 22.

13. The cyanobacterial cell of claim 1, wherein the yqhD gene encodes a polypeptide having at least 98% identity to SEQ ID NO: 23.

14. The cyanobacterial cell of claim 1, wherein at least one of said DAR1, GPP2, dhaB1-3, orfZ, orf2b, and yqhD genes is present in a separate genetic region in the cell.

15. The cyanobacterial cell of claim 14, wherein said separate genetic region in the cell is a different plasmid vector or a different chromosome.

16. The cyanobacterial cell of claim 1, wherein said cyanobacterial cell is selected from the group consisting of Synechocystis, Synechococcus, Acaryochloris, Anabaena, thermosynechococcus, Chamaesiphon, Chroococcus, Cyanobacterium, Cyanobium, Dactylococcopsis, Gloeobacter, Gloeocapsa, Gloeothece, Microcystis, Prochlorococcus, Prochloron, Chroococcidiopsis, Cyanocystis, Dermocarpella, Myxosarcina, Pleurocapsa, Stanieria, Xenococcus, Arthrospira, Borzia, Crinalium, Geitlerinema, Halospirulina, Leptolyngbya, Limnothrix, Lyngbya, Microcoleus, Cyanodictyon, Aphanocapsa, Oscillatoria, Planktothrix, Prochlorothrix, Pseudanabaena, Spirulina, Starria, Symploca, Trichodesmium, Tychonema, Anabaenopsis, Aphanizomenon, Calothrix, Cyanospira, Cylindrospermopsis, Cylindrospermum, Nodularia, Nostoc, Chlorogloeopsis, Fischerella, Geitleria, Nostochopsis, Iyengariella, Stigonema, Rivularia, Scytonema, Tolypothrix, Cyanothece, Phormidium, and Adrianema.

17. The cyanobacterial cell of claim 1, wherein said cyanobacterial cell is selected from the group consisting of Synechocystis sp. PCC 6803 and Synechococcus sp. PCC 7002.

18. A method of producing 1,3-propanediol in a cyanobacterial cell, comprising:
a) introducing a nucleic acid sequence comprising a gene encoding a DAR1 enzyme, a gene encoding a GPP2 enzyme, genes encoding the DhaB1-3 enzymes, a gene encoding an OrfZ enzyme, a gene encoding an Orf2b enzyme, and a gene encoding a YqhD enzyme to a cyanobacterial cell; and
b) culturing said cyanobacterial cell under conditions which produce 1,3-propanediol.

19. A genetically enhanced Synechocystis host cell, comprising at least one promoter operatively linked to a DAR1 gene and a GPP2 gene.

20. The genetically enhanced Synechocystis host cell of claim 19, wherein said DAR1 gene has at least 98% identity to SEQ ID NO: 6 and said GPP2 gene has at least 98% identity to SEQ ID NO: 8.

21. A genetically enhanced Synechococcus host cell, comprising at least one promoter operatively linked to genes encoding dhaB1-3, orfZ, orf2b, and yqhD.

22. A method of making 1,3-propanediol, comprising growing a host cyanobacterial cell comprising at least one promoter operatively linked to the genes dhaB1-3, orfZ, and yqhD in a culture medium comprising 1-2% glycerol, wherein 1,3-propanediol is produced.

23. The method of claim 22, wherein the host cyanobacterial cell is a Synechococcus cyanobacterial cell.


SEQUENCE LISTING (41 sequences)

SEQ ID NO: 1 - DNA, 41 bp, Artificial Sequence
  Synthetic Promoter Sequence
ggcgaattgacattgtgagcggataacaatataatgtgtgg

SEQ ID NO: 2 - DNA, 604 bp, Nostoc sp. PCC7120
gataacatcaccgtcgttatcgtcgctttagaataacgttcccaaaatagctcatttccaactggcaactcacaaccaaaaaccgcatttttagtaaatatactcagcaatttgttcaacctgagcatttttcccatttgcaacttgatacaaatatttttagcagcaaattttcctactgccagcttagtttacataaattttgtctgttgacatcttgcacacaataaggtatggcgcatataatgcgatattactaccattaatttactacctagtcattaacgtctcccgccagagaacagttttgaataggtagtcaattttaggtattgaacctgctgtaaatttattaaatcgatgaatttccccgaaatctgctctagcagacttgggttatataccagtaggctcaggtgcaaaacaacaaagcacaaattttacccattaaggatataggcaatctgtcaaatagttgttatctttcttaatacagaggaataatcaacaatatggggcaggtactaactaaagtcctatgcctgtggggcttctgtaaccgacataacctttacgcgttgtcttttaggagtctgttatgaac

SEQ ID NO: 3 - DNA, 267 bp, Synechocystis sp. PCC6803
tcgacatcaggaattgtaattagaaagtccaaaaattgtaatttaaaaaacagtcaatggagagcattgccataagtaaaggcatcccctgcgtgataagattaccttcagaaaacagatagttgctgggttatcgcagatttttctcgcaaccaaataactgtaaataataactgtctctggggcgacggtaggctttatattgccaaatttcgcccgtgggagaaagctaggctattcaatgtttatggaggactgacctagatg

SEQ ID NO: 4 - DNA, 123 bp, Synechococcus sp. PCC 7002
tcgactgtggtctgtctttgttcgctgatctaaacaatacctgaataattgttcatgtgttaatctaaaaatgtgaacaatcgttcaactatttaagacaataccttggaggtttaaaccatg

SEQ ID NO: 5 - DNA, 550 bp, Synechocystis sp. PCC 6803
ggcggccaacgtgatttaaagaaaaacctccttgaaccgtagcacaaatcttgaaacacctgaagatatgctcagatattaaagatgttaggatgaaaatcattttctaaatccagtttaaatttttccctagctcctaacgccaacctctatgagtaagtcctcgttgtcaaagtcacaatcctgccagaacgaagagatgcccctttgtgatcaacctcttgttcatcttgagcaggtacgacaggttcaaccagaggtgatgtcattggaccaggcccagcaaatggcggagtttttcagtgcactagctgatccgagtcggttgcgtttaatgtcggcattggcccgccaagaactctgtgtctgtgatttagcagcggcgatgaaagtgagtgaatcggcagtttcccatcaattacgaattttacgatcgcagcgcctggtaaagtatcgccgggtcggccgtaatgtttactacagcttggcggataatcatgtgatgaatttgtatcgggaagttgcagaccatttgcaggaatcggattaa

SEQ ID NO: 6 - DNA, 1176 bp, Saccharomyces cerevisiae
atgtctgctgctgctgatagattaaacttaacttccggccacttgaatgctggtagaaagagaagttcctcttctgtttctttgaaggctgccgaaaagcctttcaaggttactgtgattggatctggtaactggggtactactattgccaaggtggttgccgaaaattgtaagggatacccagaagttttcgctccaatagtacaaatgtgggtgttcgaagaagagatcaatggtgaaaaattgactgaaatcataaatactagacatcaaaacgtgaaatacttgcctggcatcactctacccgacaatttggttgctaatccagacttgattgattcagtcaaggatgtcgacatcatcgttttcaacattccacatcaatttttgccccgtatctgtagccaattgaaaggtcatgttgattcacacgtcagagctatctcctgtctaaagggttttgaagttggtgctaaaggtgtccaattgctatcctcttacatcactgaggaactaggtattcaatgtggtgctctatctggtgctaacattgccaccgaagtcgctcaagaacactggtctgaaacaacagttgcttaccacattccaaaggatttcagaggcgagggcaaggacgtcgaccataaggttctaaaggccttgttccacagaccttacttccacgttagtgtcatcgaagatgttgctggtatctccatctgtggtgctttgaagaacgttgttgccttaggttgtggtttcgtcgaaggtctaggctggggtaacaacgcttctgctgccatccaaagagtcggtttgggtgagatcatcagattcggtcaaatgtttttcccagaatctagagaagaaacatactaccaagagtctgctggtgttgctgatttgatcaccacctgcgctggtggtagaaacgtcaaggttgctaggctaatggctacttctggtaaggacgcctgggaatgtgaaaaggagttgttgaatggccaatccgctcaaggtttaattacctgcaaagaagttcacgaatggttggaaacatgtggctctgtcgaagacttcccattatttgaagccgtataccaaatcgtttacaacaactacccaatgaagaacctgccggacatgattgaagaattagatctacatgaagattag

SEQ ID NO: 7 - protein, 391 aa, Saccharomyces cerevisiae
MSAAADRLNLTSGHLNAGRKRSSSSVSLKAAEKPFKVTVIGSGNWGTTIAKVVAENCKGYPEVFAPIVQMWVFEEEINGEKLTEIINTRHQNVKYLPGITLPDNLVANPDLIDSVKDVDIIVFNIPHQFLPRICSQLKGHVDSHVRAISCLKGFEVGAKGVQLLSSYITEELGIQCGALSGANIATEVAQEHWSETTVAYHIPKDFRGEGKDVDHKVLKALFHRPYFHVSVIEDVAGISICGALKNVVALGCGFVEGLGWGNNASAAIQRVGLGEIIRFGQMFFPESREETYYQESAGVADLITTCAGGRNVKVARLMATSGKDAWECEKELLNGQSAQGLITCKEVHEWLETCGSVEDFPLFEAVYQIVYNNYPMKNLPDMIEELDLHED

SEQ ID NO: 8 - DNA, 753 bp, Saccharomyces cerevisiae
atgggattgactactaaacctctatctttgaaagttaacgccgctttgttcgacgtcgacggtaccattatcatctctcaaccagccattgctgcattctggagggatttcggtaaggacaaaccttatttcgatgctgaacacgttatccaagtctcgcatggttggagaacgtttgatgccattgctaagttcgctccagactttgccaatgaagagtatgttaacaaattagaagctgaaattccggtcaagtacggtgaaaaatccattgaagtcccaggtgcagttaagctgtgcaacgctttgaacgctctaccaaaagagaaatgggctgtggcaacttccggtacccgtgatatggcacaaaaatggttcgagcatctgggaatcaggagaccaaagtacttcattaccgctaatgatgtcaaacagggtaagcctcatccagaaccatatctgaagggcaggaatggcttaggatatccgatcaatgagcaagacccttccaaatctaaggtagtagtatttgaagacgctccagcaggtattgccgccggaaaagccgccggttgtaagatcattggtattgccactactttcgacttggacttcctaaaggaaaaaggctgtgacatcattgtcaaaaaccacgaatccatcagagttggcggctacaatgccgaaacagacgaagttgaattcatttttgacgactacttatatgctaaggacgatctgttgaaatggtaa

SEQ ID NO: 9 - protein, 250 aa, Saccharomyces cerevisiae
MGLTTKPLSLKVNAALFDVDGTIIISQPAIAAFWRDFGKDKPYFDAEHVIQVSHGWRTFDAIAKFAPDFANEEYVNKLEAEIPVKYGEKSIEVPGAVKLCNALNALPKEKWAVATSGTRDMAQKWFEHLGIRRPKYFITANDVKQGKPHPEPYLKGRNGLGYPINEQDPSKSKVVVFEDAPAGIAAGKAAGCKIIGIATTFDLDFLKEKGCDIIVKNHESIRVGGYNAETDEVEFIFDDYLYAKDDLLKW

SEQ ID NO: 10 - DNA, 2693 bp, Klebsiella pneumonia
atgaaaagatcaaaacgatttgcagtactggcccagcgccccgtcaatcaggacgggctgattggcgagtggcctgaagaggggctgatcgccatggacagcccctttgacccggtctcttcagtaaaagtggacaacggtctgatcgtcgagctggacggcaaacgccgggaccagtttgacatgatcgaccggtttatcgccgattacgcgatcaacgttgagcgcacagagcaggcaatgcgcctggaggcggtggaaatagcccgcatgctggtggatattcacgtcagccgggaggagatcattgccatcactaccgccatcacgccggccaaagcggtcgaggtgatggcgcagatgaacgtggtggagatgatgatggcgctgcagaagatgcgtgcccgccggaccccctccaaccagtgccacgtcaccaatctcaaagataatccggtgcagattgccgctgacgccgccgaggccgggatccgcggcttctcagaacaggagaccacggtcggtatcgcgcgctatgcgccgtttaacgccctggcgctgttggtcggctcgcagtgcggccgtcccggcgtgttgacgcagtgctcggtggaagaggccaccgagctggagctgggcatgcgtggcttaaccagctacgccgagacggtgtcggtctacggcactgaagcggtatttaccgacggcgatgatactccgtggtcaaaggcgttcctcgcctcggcctacgcctcccgcgggttgaaaatgcgctacacctccggcaccggatccgaagcgctgatgggctattcggagagcaagtcgatgctctacctcgaatcgcgctgcatcttcattaccaaaggcgccggggttcaggggctgcaaaacggcgcagtgagctgtatcggcatgaccggcgctgtgccgtcgggcattcgggcggtgctggcggaaaacctgatcgcctctatgctcgacctcgaagtggcgtccgccaacgaccagactttctcccactcggatattcgccgcaccgcgcgcaccctgatgcagatgctgccgggcaccgactttattttctccggctacagcgcggtgccgaactacgacaacatgttcgccggctcgaacttcgatgcggaagattttgatgattacaacattctgcagcgtgacctgatggttgacggcggcctgcgtccggtgaccgaggcggaaaccattgccattcgccagaaagcggcgcgggcgatccaggcggttttccgcgagctggggctgccgccaatcgccgacgaggaggtggaggccgccacctacgcgcacggcagcaacgagatgccgccgcgtaacgtggtggaggatctgagtgcggtggaagagatgatgaagcgcaacatcaccggcctcgatattgtcggcgcgctgagccgcagcggctttgaggatatcgccagcaatattctcaatatgctgcgccagcgggtcaccggcgattacctgcagacctcggccattctcgatcgacagttcgaggtggtgagcgcggtcaacgacatcaatgactatcaggggccgggcaccggctatcgcatctctgccgaacgctgggcggagatcaaaaatattccgggcgtggttcagcctgacaccattgaataaggcggtattcctgtgcaacagacaactcaaattcagccctcttttaccctgaaaacccgcgagggcggggtagcttctgccgatgaacgtgccgatgaagtggtgatcggcgtcggccctgccttcgataaacaccagcatcacactctgatcgatatgccccatggcgcgatcctcaaagagctgattgccggggtggaagaagaggggcttcacgcccgggtggtgcgcattctgcgcacgtccgacgtctcctttatggcctgggatgcggccaacctgagcggctcggggatcggcatcggtatccagtcgaaggggaccacggtcatccatcagcgcgatctgctgccgctcagcaacctggagctgttctcccaggcgccgctgctgacgctggagacctaccggcagattggcaaaaacgccgcgcgctatgcgcgcaaagagtcaccttcgccggtgccggtggtgaacgatcagatggtgcggccgaaatttatggccaaagccgcgctatttcatatcaaagagaccaaacatgtggtgcaggacgccgagcccgtcaccctgcacgtcgacttagtaagggagtgaccatgagcgagaaaaccatgcgcgtgcaggattatccgttagccacccgctgcccggagcatatcctgacgcctaccggcaaaccattgaccgatattaccctcgagaaggtgctctctggcgaggtgggcccgcaggatgtgcggatctcccgccagacccttgagtaccaggcgcagattgccgagcagatgcagcgccatgcggtggcgcgcaatttccgccgcgcggcggagcttatcgccattcctgacgagcgcattctggctatctataacgcgctgcgcccgttccgctcctcgcaggcggagctgctggcgatcgccgacgagctggagcacacctggcatgcgacagtgaatgccgcctttgtccgggagtcggcggaagtgtatcagcagcggcataagctgcgtaaaggaagctaa

SEQ ID NO: 11 - DNA, 1668 bp, Klebsiella pneumonia
atgaaaagatcaaaacgatttgcagtactggcccagcgccccgtcaatcaggacgggctgattggcgagtggcctgaagaggggctgatcgccatggacagcccctttgacccggtctcttcagtaaaagtggacaacggtctgatcgtcgagctggacggcaaacgccgggaccagtttgacatgatcgaccggtttatcgccgattacgcgatcaacgttgagcgcacagagcaggcaatgcgcctggaggcggtggaaatagcccgcatgctggtggatattcacgtcagccgggaggagatcattgccatcactaccgccatcacgccggccaaagcggtcgaggtgatggcgcagatgaacgtggtggagatgatgatggcgctgcagaagatgcgtgcccgccggaccccctccaaccagtgccacgtcaccaatctcaaagataatccggtgcagattgccgctgacgccgccgaggccgggatccgcggcttctcagaacaggagaccacggtcggtatcgcgcgctatgcgccgtttaacgccctggcgctgttggtcggctcgcagtgcggccgtcccggcgtgttgacgcagtgctcggtggaagaggccaccgagctggagctgggcatgcgtggcttaaccagctacgccgagacggtgtcggtctacggcactgaagcggtatttaccgacggcgatgatactccgtggtcaaaggcgttcctcgcctcggcctacgcctcccgcgggttgaaaatgcgctacacctccggcaccggatccgaagcgctgatgggctattcggagagcaagtcgatgctctacctcgaatcgcgctgcatcttcattaccaaaggcgccggggttcaggggctgcaaaacggcgcagtgagctgtatcggcatgaccggcgctgtgccgtcgggcattcgggcggtgctggcggaaaacctgatcgcctctatgctcgacctcgaagtggcgtccgccaacgaccagactttctcccactcggatattcgccgcaccgcgcgcaccctgatgcagatgctgccgggcaccgactttattttctccggctacagcgcggtgccgaactacgacaacatgttcgccggctcgaacttcgatgcggaagattttgatgattacaacattctgcagcgtgacctgatggttgacggcggcctgcgtccggtgaccgaggcggaaaccattgccattcgccagaaagcggcgcgggcgatccaggcggttttccgcgagctggggctgccgccaatcgccgacgaggaggtggaggccgccacctacgcgcacggcagcaacgagatgccgccgcgtaacgtggtggaggatctgagtgcggtggaagagatgatgaagcgcaacatcaccggcctcgatattgtcggcgcgctgagccgcagcggctttgaggatatcgccagcaatattctcaatatgctgcgccagcgggtcaccggcgattacctgcagacctcggccattctcgatcgacagttcgaggtggtgagcgcggtcaacgacatcaatgactatcaggggccgggcaccggctatcgcatctctgccgaacgctgggcggagatcaaaaatattccgggcgtggttcagcctgacaccattgaataa

SEQ ID NO: 12 - protein, 555 aa, Klebsiella pneumonia
MKRSKRFAVLAQRPVNQDGLIGEWPEEGLIAMDSPFDPVSSVKVDNGLIVELDGKRRDQFDMIDRFIADYAINVERTEQAMRLEAVEIARMLVDIHVSREEIIAITTAITPAKAVEVMAQMNVVEMMMALQKMRARRTPSNQCHVTNLKDNPVQIAADAAEAGIRGFSEQETTVGIARYAPFNALALLVGSQCGRPGVLTQCSVEEATELELGMRGLTSYAETVSVYGTEAVFTDGDDTPWSKAFLASAYASRGLKMRYTSGTGSEALMGYSESKSMLYLESRCIFITKGAGVQGLQNGAVSCIGMTGAVPSGIRAVLAENLIASMLDLEVASANDQTFSHSDIRRTARTLMQMLPGTDFIFSGYSAVPNYDNMFAGSNFDAEDFDDYNILQRDLMVDGGLRPVTEAETIAIRQKAARAIQAVFRELGLPPIADEEVEAATYAHGSNEMPPRNVVEDLSAVEEMMKRNITGLDIVGALSRSGFEDIASNILNMLRQRVTGDYLQTSAILDRQFEVVSAVNDINDYQGPGTGYRISAERWAEIKNIPGVVQPDTIE

SEQ ID NO: 13 - DNA, 585 bp, Klebsiella pneumonia
gtgcaacagacaactcaaattcagccctcttttaccctgaaaacccgcgagggcggggtagcttctgccgatgaacgtgccgatgaagtggtgatcggcgtcggccctgccttcgataaacaccagcatcacactctgatcgatatgccccatggcgcgatcctcaaagagctgattgccggggtggaagaagaggggcttcacgcccgggtggtgcgcattctgcgcacgtccgacgtctcctttatggcctgggatgcggccaacctgagcggctcggggatcggcatcggtatccagtcgaaggggaccacggtcatccatcagcgcgatctgctgccgctcagcaacctggagctgttctcccaggcgccgctgctgacgctggagacctaccggcagattggcaaaaacgccgcgcgctatgcgcgcaaagagtcaccttcgccggtgccggtggtgaacgatcagatggtgcggccgaaatttatggccaaagccgcgctatttcatatcaaagagaccaaacatgtggtgcaggacgccgagcccgtcaccctgcacgtcgacttagtaagggagtga

SEQ ID NO: 14 - protein, 194 aa, Klebsiella pneumonia
VQQTTQIQPSFTLKTREGGVASADERADEVVIGVGPAFDKHQHHTLIDMPHGAILKELIAGVEEEGLHARVVRILRTSDVSFMAWDAANLSGSGIGIGIQSKGTTVIHQRDLLPLSNLELFSQAPLLTLETYRQIGKNAARYARKESPSPVPVVNDQMVRPKFMAKAALFHIKETKHVVQDAEPVTLHVDLVRE

SEQ ID NO: 15 - DNA, 426 bp, Klebsiella pneumonia
atgagcgagaaaaccatgcgcgtgcaggattatccgttagccacccgctgcccggagcatatcctgacgcctaccggcaaaccattgaccgatattaccctcgagaaggtgctctctggcgaggtgggcccgcaggatgtgcggatctcccgccagacccttgagtaccaggcgcagattgccgagcagatgcagcgccatgcggtggcgcgcaatttccgccgcgcggcggagcttatcgccattcctgacgagcgcattctggctatctataacgcgctgcgcccgttccgctcctcgcaggcggagctgctggcgatcgccgacgagctggagcacacctggcatgcgacagtgaatgccgcctttgtccgggagtcggcggaagtgtatcagcagcggcataagctgcgtaaaggaagctaa

SEQ ID NO: 16 - protein, 141 aa, Klebsiella pneumonia
MSEKTMRVQDYPLATRCPEHILTPTGKPLTDITLEKVLSGEVGPQDVRISRQTLEYQAQIAEQMQRHAVARNFRRAAELIAIPDERILAIYNALRPFRSSQAELLAIADELEHTWHATVNAAFVRESAEVYQQRHKLRKGS

SEQ ID NO: 17 - DNA, 2202 bp, Klebsiella pneumonia
atgccgttaatagccgggattgatatcggcaacgccaccaccgaggtggcgctggcgtccgatgacccgcaggcgagggcgtttgttgccagcgggatcgtcgcgacgacgggcatgaaagggacgcgggacaatatcgccgggaccctcgccgcgctggagcaggccctggcgaaaacaccgtggtcgatgagcgatgtctctcgcatctatcttaacgaagccgcgccggtgattggcgatgtggcgatggagaccatcaccgagaccattatcaccgaatcgaccatgatcggtcataacccgcagacgccgggcggggtgggcgttggcgtggggacgactatcgccctcgggcggctggcgacgctgccggcggcgcagtatgccgaggggtggatcgtactgattgacgacgccgtcgatttccttgacgccgtgtggtggctcaatgaggcgctcgaccgggggatcaacgtggtggcggcgatcctcaaaaaggacgacggcgtgctggtgaacaaccgcctgcgtaaaaccctgccggtggtggatgaagtgacgctgctggagcaggtccccgagggggtaatggcggcggtggaagtggccgcgccgggccaggtggtgcggatcctgtcgaatccctacgggatcgccaccttcttcgggctaagcccggaagagacccaggccatcgtccccatcgcccgcgccctgattggcaaccgttcagcggtggtgctcaagaccccgcagggggatgtgcagtcgcgggtgatcccggcgggcaacctctacattagcggcgaaaagcgccgcggagaggccgatgtcgccgagggcgcggaagccatcatgcaggcgatgagcgcctgcgctccggtacgcgacatccgcggcgaaccgggcacccacgccggcggcatgcttgagcgggtgcgcaaggtaatggcgtccctgaccggccatgagatgagcgcgatatacatccaggatctgctggcggtggatacgtttattccgcgcaaggtgcagggcgggatggccggcgagtgcgccatggagaatgccgtcgggatggcggcgatggtgaaagcggatcgtctgcaaatgcaggttatcgcccgcgaactgagcgcccgactgcagaccgaggtggtggtgggcggcgtggaggccaacatggccatcgccggggcgttaaccactcccggctgtgcggcgccgctggcgatcctcgacctcggcgccggctcgacggatgcggcgatcgtcaacgcggaggggcagataacggcggtccatctcgccggggcggggaatatggtcagcctgttgattaaaaccgagctgggcctcgaggatctttcgctggcggaagcgataaaaaaatacccgctggccaaagtggaaagcctgttcagtattcgtcacgagaatggcgcggtggagttctttcgggaagccctcagcccggcggtgttcgccaaagtggtgtacatcaaggagggcgaactggtgccgatcgataacgccagcccgctggaaaaaattcgtctcgtgcgccggcaggcgaaagagaaagtgtttgtcaccaactgcctgcgcgcgctgcgccaggtctcacccggcggttccattcgcgatatcgcctttgtggtgctggtgggcggctcatcgctggactttgagatcccgcagcttatcacggaagccttgtcgcactatggcgtggtcgccgggcagggcaatattcggggaacagaagggccgcgcaatgcggtcgccaccgggctgctactggccggtcaggcgaattaaggcgcgccaagaggagaactagtaatgtcgctttcaccgccaggcgtacgcctgttttacgatccgcgcgggcaccatgccggcgccatcaatgagctgtgctgggggctggaggagcagggggtcccctgccagaccataacctatgacggaggcggtgacgccgctgcgctgggcgccctggcggccagaagctcgcccctgcgggtgggtatcgggctcagcgcgtccggcgagatagccctcactcatgcccagctgccggcggacgcgccgctggctaccggacacgtcaccgatagcgacgatcatctgcgtacgctcggcgccaacgccgggcagctggttaaagtcctgccgttaagtgagagaaactga

SEQ ID NO: 18 - DNA, 1824 bp, Klebsiella pneumonia
atgccgttaatagccgggattgatatcggcaacgccaccaccgaggtggcgctggcgtccgatgacccgcaggcgagggcgtttgttgccagcgggatcgtcgcgacgacgggcatgaaagggacgcgggacaatatcgccgggaccctcgccgcgctggagcaggccctggcgaaaacaccgtggtcgatgagcgatgtctctcgcatctatcttaacgaagccgcgccggtgattggcgatgtggcgatggagaccatcaccgagaccattatcaccgaatcgaccatgatcggtcataacccgcagacgccgggcggggtgggcgttggcgtggggacgactatcgccctcgggcggctggcgacgctgccggcggcgcagtatgccgaggggtggatcgtactgattgacgacgccgtcgatttccttgacgccgtgtggtggctcaatgaggcgctcgaccgggggatcaacgtggtggcggcgatcctcaaaaaggacgacggcgtgctggtgaacaaccgcctgcgtaaaaccctgccggtggtggatgaagtgacgctgctggagcaggtccccgagggggtaatggcggcggtggaagtggccgcgccgggccaggtggtgcggatcctgtcgaatccctacgggatcgccaccttcttcgggctaagcccggaagagacccaggccatcgtccccatcgcccgcgccctgattggcaaccgttcagcggtggtgctcaagaccccgcagggggatgtgcagtcgcgggtgatcccggcgggcaacctctacattagcggcgaaaagcgccgcggagaggccgatgtcgccgagggcgcggaagccatcatgcaggcgatgagcgcctgcgctccggtacgcgacatccgcggcgaaccgggcacccacgccggcggcatgcttgagcgggtgcgcaaggtaatggcgtccctgaccggccatgagatgagcgcgatatacatccaggatctgctggcggtggatacgtttattccgcgcaaggtgcagggcgggatggccggcgagtgcgccatggagaatgccgtcgggatggcggcgatggtgaaagcggatcgtctgcaaatgcaggttatcgcccgcgaactgagcgcccgactgcagaccgaggtggtggtgggcggcgtggaggccaacatggccatcgccggggcgttaaccactcccggctgtgcggcgccgctggcgatcctcgacctcggcgccggctcgacggatgcggcgatcgtcaacgcggaggggcagataacggcggtccatctcgccggggcggggaatatggtcagcctgttgattaaaaccgagctgggcctcgaggatctttcgctggcggaagcgataaaaaaatacccgctggccaaagtggaaagcctgttcagtattcgtcacgagaatggcgcggtggagttctttcgggaagccctcagcccggcggtgttcgccaaagtggtgtacatcaaggagggcgaactggtgccgatcgataacgccagcccgctggaaaaaattcgtctcgtgcgccggcaggcgaaagagaaagtgtttgtcaccaactgcctgcgcgcgctgcgccaggtctcacccggcggttccattcgcgatatcgcctttgtggtgctggtgggcggctcatcgctggactttgagatcccgcagcttatcacggaagccttgtcgcactatggcgtggtcgccgggcagggcaatattcggggaacagaagggccgcgcaatgcggtcgccaccgggctgctactggccggtcaggcgaattaa

SEQ ID NO: 19 - protein, 607 aa, Klebsiella pneumonia
MPLIAGIDIGNATTEVALASDDPQARAFVASGIVATTGMKGTRDNIAGTLAALEQALAKTPWSMSDVSRIYLNEAAPVIGDVAMETITETIITESTMIGHNPQTPGGVGVGVGTTIALGRLATLPAAQYAEGWIVLIDDAVDFLDAVWWLNEALDRGINVVAAILKKDDGVLVNNRLRKTLPVVDEVTLLEQVPEGVMAAVEVAAPGQVVRILSNPYGIATFFGLSPEETQAIVPIARALIGNRSAVVLKTPQGDVQSRVIPAGNLYISGEKRRGEADVAEGAEAIMQAMSACAPVRDIRGEPGTHAGGMLERVRKVMASLTGHEMSAIYIQDLLAVDTFIPRKVQGGMAGECAMENAVGMAAMVKADRLQMQVIARELSARLQTEVVVGGVEANMAIAGALTTPGCAAPLAILDLGAGSTDAAIVNAEGQITAVHLAGAGNMVSLLIKTELGLEDLSLAEAIKKYPLAKVESLFSIRHENGAVEFFREALSPAVFAKVVYIKEGELVPIDNASPLEKIRLVRRQAKEKVFVTNCLRALRQVSPGGSIRDIAFVVLVGGSSLDFEIPQLITEALSHYGVVAGQGNIRGTEGPRNAVATGLLLAGQAN

SEQ ID NO: 20 - DNA, 354 bp, Klebsiella pneumonia
atgtcgctttcaccgccaggcgtacgcctgttttacgatccgcgcgggcaccatgccggcgccatcaatgagctgtgctgggggctggaggagcagggggtcccctgccagaccataacctatgacggaggcggtgacgccgctgcgctgggcgccctggcggccagaagctcgcccctgcgggtgggtatcgggctcagcgcgtccggcgagatagccctcactcatgcccagctgccggcggacgcgccgctggctaccggacacgtcaccgatagcgacgatcatctgcgtacgctcggcgccaacgccgggcagctggttaaagtcctgccgttaagtgagagaaactga

SEQ ID NO: 21 - protein, 117 aa, Klebsiella pneumonia
MSLSPPGVRLFYDPRGHHAGAINELCWGLEEQGVPCQTITYDGGGDAAALGALAARSSPLRVGIGLSASGEIALTHAQLPADAPLATGHVTDSDDHLRTLGANAGQLVKVLPLSERN

SEQ ID NO: 22 - DNA, 1164 bp, Escherichia coli
atgaacaactttaatctgcacaccccaacccgcattctgtttggtaaaggcgcaatcgctggtttacgcgaacaaattcctcacgatgctcgcgtattgattacctacggcggcggcagcgtgaaaaaaaccggcgttctcgatcaagttctggatgccctgaaaggcatggacgtgctggaatttggcggtattgagccaaacccggcttatgaaacgctgatgaacgccgtgaaactggttcgcgaacagaaagtgactttcctgctggcggttggcggcggttctgtactggacggcaccaaatttatcgccgcagcggctaactatccggaaaatatcgatccgtggcacattctgcaaacgggcggtaaagagattaaaagcgccatcccgatgggctgtgtgctgacgctgccagcaaccggttcagaatccaacgcaggcgcggtgatctcccgtaaaaccacaggcgacaagcaggcgttccattctgcccatgttcagccggtatttgccgtgctcgatccggtttatacctacaccctgccgccgcgtcaggtggctaacggcgtagtggacgcctttgtacacaccgtggaacagtatgttaccaaaccggttgatgccaaaattcaggaccgtttcgcagaaggcattttgctgacgctaatcgaagatggtccgaaagccctgaaagagccagaaaactacgatgtgcgcgccaacgtcatgtgggcggcgactcaggcgctgaacggtttgattggcgctggcgtaccgcaggactgggcaacgcatatgctgggccacgaactgactgcgatgcacggtctggatcacgcgcaaacactggctatcgtcctgcctgcactgtggaatgaaaaacgcgataccaagcgcgctaagctgctgcaatatgctgaacgcgtctggaacatcactgaaggttccgatgatgagcgtattgacgccgcgattgccgcaacccgcaatttctttgagcaattaggcgtgccgacccacctctccgactacggtctggacggcagctccatcccggctttgctgaaaaaactggaagagcacggcatgacccaactgggcgaaaatcatgacattacgttggatgtcagccgccgtatatacgaagccgcccgctaa

SEQ ID NO: 23 - protein, 387 aa, Escherichia coli
MNNFNLHTPTRILFGKGAIAGLREQIPHDARVLITYGGGSVKKTGVLDQVLDALKGMDVLEFGGIEPNPAYETLMNAVKLVREQKVTFLLAVGGGSVLDGTKFIAAAANYPENIDPWHILQTGGKEIKSAIPMGCVLTLPATGSESNAGAVISRKTTGDKQAFHSAHVQPVFAVLDPVYTYTLPPRQVANGVVDAFVHTVEQYVTKPVDAKIQDRFAEGILLTLIEDGPKALKEPENYDVRANVMWAATQALNGLIGAGVPQDWATHMLGHELTAMHGLDHAQTLAIVLPALWNEKRDTKRAKLLQYAERVWNITEGSDDERIDAAIAATRNFFEQLGVPTHLSDYGLDGSSIPALLKKLEEHGMTQLGENHDITLDVSRRIYEAAR

SEQ ID NO: 24 - DNA, 56 bp, Artificial Sequence
  Synthetic primer sequence
gtcaatcccatatgtagatctcctgaattcctaatcttcatgtagatctaattctt

SEQ ID NO: 25 - DNA, 52 bp, Artificial Sequence
  Synthetic primer sequence
aggagtctgttatgaacggtaccatgaattcatgtctgctgctgctgataga

SEQ ID NO: 26 - DNA, 52 bp, Artificial Sequence
  Synthetic primer sequence
atgtttatggaggactgacctagatgaattcatgtctgctgctgctgataga

SEQ ID NO: 27 - DNA, 53 bp, Artificial Sequence
  Synthetic primer sequence
atgaagattaggaattcaggagatctacatatgggattgactactaaacctct

SEQ ID NO: 28 - DNA, 53 bp, Artificial Sequence
  Synthetic primer sequence
gatcttttcatcctgcaggctcctgaattcttaccatttcaacagatcgtcct

SEQ ID NO: 29 - DNA, 53 bp, Artificial Sequence
  Synthetic primer sequence
tgaaatggtaagaattcaggagcctgcaggatgaaaagatcaaaacgatttgc

SEQ ID NO: 30 - DNA, 72 bp, Artificial Sequence
  Synthetic primer sequence
aatgtgtggatcagcaggacgcactgaccggaattcaggagcctgcaggatgaaaagatcaaaacgatttgc

SEQ ID NO: 31 - DNA, 49 bp, Artificial Sequence
  Synthetic primer sequence
gttcatcgctagctctcctcttggcgcgccttaattcgcctgaccggcc

SEQ ID NO: 32 - DNA, 19 bp, Artificial Sequence
  Synthetic primer sequence
gcaggcggagctgctggcg

SEQ ID NO: 33 - DNA, 54 bp, Artificial Sequence
  Synthetic primer sequence
aattaaggcgcgccaagaggagagctagcgatgaacaactttaatctgcacacc

SEQ ID NO: 34 - DNA, 60 bp, Artificial Sequence
  Synthetic primer sequence
cgctactgccgccaggcaaattctgtttcctgcaggcgcgccgcttagcgggcggcttcg

SEQ ID NO: 35 - DNA, 60 bp, Artificial Sequence
  Synthetic primer sequence
ctagagcatgcagatctagcggccgctcgatgcaggcgcgccgcttagcgggcggcttcg

SEQ ID NO: 36 - DNA, 24 bp, Artificial Sequence
  Synthetic primer sequence
actgttccacggtgtgtacaaagg

SEQ ID NO: 37 - DNA, 55 bp, Artificial Sequence
  Synthetic primer sequence
gtcaggcgaattaaggcgcgccaggagaactagtaatgtcgctttcaccgccagg

SEQ ID NO: 38 - DNA, 45 bp, Artificial Sequence
  Synthetic primer sequence
gctagctctcctcttggcgcgcctcagtttctctcacttaacggc

SEQ ID NO: 39 - DNA, 11131 bp, Artificial Sequence
  Synthetic plasmid vector sequence containing DAR1 and GPP2
cgaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataataacgatctgatagagaagggtttgctcgggtcggtggctctggtaacgaccagtatcccgatcccggctggccgtcctggccgccacatgaggcatgttccgcgtccttgcaatactgtgtttacatacagtctatcgcttagcggaaagttcttttaccctcagccgaaatgcctgccgttgctagacattgccagccagtgcccgtcactcccgtactaactgtcacgaacccctgcaataactgtcacgcccccctgcaataactgtcacgaacccctgcaataactgtcacgcccccaaacctgcaaacccagcaggggcgggggctggcggggtgttggaaaaatccatccatgattatctaagaataatccactaggcgcggttatcagcgcccttgtggggcgctgctgcccttgcccaatatgcccggccagaggccggatagctggtctattcgctgcgctaggctacacaccgccccaccgctgcgcggcagggggaaaggcgggcaaagcccgctaaaccccacaccaaaccccgcagaaatacgctggagcgcttttagccgctttagcggcctttccccctacccgaagggtgggggcgcgtgtgcagccccgcagggcctgtctcggtcgatcattcagcccggctcatccttctggcgtggcggcagaccgaacaaggcgcggtcgtggtcgcgttcaaggtacgcatccattgccgccatgagccgatcctccggccactcgctgctgttcaccttggccaaaatcatggcccccaccagcaccttgcgccttgtttcgttcttgcgctcttgctgctgttcccttgcccgcacccgctgaatttcggcattgattcgcgctcgttgttcttcgagcttggccagccgatccgccgccttgttgctccccttaaccatcttgacaccccattgttaatgtgctgtctcgtaggctatcatggaggcacagcggcggcaatcccgaccctactttgtaggggagggcgcacttaccggtttctcttcgagaaactggcctaacggccacccttcgggcggtgcgctctccgagggccattgcatggagccgaaaagcaaaagcaacagcgaggcagcatggcgatttatcaccttacggcgaaaaccggcagcaggtcgggcggccaatcggccagggccaaggccgactacatccagcgcgaaggcaagtatgcccgcgacatggatgaagtcttgcacgccgaatccgggcacatgccggagttcgtcgagcggcccgccgactactgggatgctgccgacctgtatgaacgcgccaatgggcggctgttcaaggaggtcgaatttgccctgccggtcgagctgaccctcgaccagcagaaggcgctggcgtccgagttcgcccagcacctgaccggtgccgagcgcctgccgtatacgctggccatccatgccggtggcggcgagaacccgcactgccacctgatgatctccgagcggatcaatgacggcatcgagcggcccgccgctcagtggttcaagcggtacaacggcaagaccccggagaagggcggggcacagaagaccgaagcgctcaagcccaaggcatggcttgagcagacccgcgaggcatgggccgaccatgccaaccgggcattagagcgggctggccacgacgcccgcattgaccacagaacacttgaggcgcagggcatcgagcgcctgcccggtgttcacctggggccgaacgtggtggagatggaaggccggggcatccgcaccgaccgggcagacgtggccctgaacatcgacaccgccaacgcccagatcatcgacttacaggaataccgggaggcaatagaccatgaacgcaatcgacagagtgaagaaatccagaggcatcaacgagttagcggagcagatcgaaccgctggcccagagcatggcgacactggccgacgaagcccggcaggtcatgagccagacccagcaggccagcgaggcgcaggcggcggagtggctgaaagcccagcgccagacaggggcggcatgggtggagctggccaaagagttgcgggaggtagccgccgaggtgagcagcgccgcgcagagcgcccggagcgcgtcgcgggggtggcactggaagctatggctaaccgtgatgctggcttccatgatgcctacggtggtgctgctgatcgcatcgttgctcttgctcgacctgacgccactgacaaccgaggacggctcgatctggctgcgcttggtggcccgatgaagaacgacaggactttgcaggccataggccgacagctcaaggccatgggctgtgagcgcttcgatatcggcgtcagggacgccaccaccggccagatgatgaaccgggaatggtcagccgccgaagtgctccagaacacgccatggctcaagcggatgaatgcccagggcaatgacgtgtatatcaggcccgccgagcaggagcggcatggtctggtgctggtggacgacctcagcgagtttgacctggatgacatgaaagccgagggccgggagcctgccctggtagtggaaaccagcccgaagaactatcaggcatgggtcaaggtggccgacgccgcaggcggtgaacttcgggggcagattgcccggacgctggccagcgagtacgacgccgacccggccagcgccgacagccgccactatggccgcttggcgggcttcaccaaccgcaaggacaagcacaccacccgcgccggttatcagccgtgggtgctgctgcgtgaatccaagggcaagaccgccaccgctggcccggcgctggtgcagcaggctggccagcagatcgagcaggcccagcggcagcaggagaaggcccgcaggctggccagcctcgaactgcccgagcggcagcttagccgccaccggcgcacggcgctggacgagtaccgcagcgagatggccgggctggtcaagcgcttcggtgatgacctcagcaagtgcgactttatcgccgcgcagaagctggccagccggggccgcagtgccgaggaaatcggcaaggccatggccgaggccagcccagcgctggcagagcgcaagcccggccacgaagcggattacatcgagcgcaccgtcagcaaggtcatgggtctgcccagcgtccagcttgcgcgggccgagctggcacgggcaccggcaccccgccagcgaggcatggacaggggcgggccagatttcagcatgtagtgcttgcgttggtactcacgcctgttatactatgagtactcacgcacagaagggggttttatggaatacgaaaaaagcgcttcagggtcggtctacctgatcaaaagtgacaagggctattggttgcccggtggctttggttatacgtcaaacaaggccgaggctggccgcttttcagtcgctgatatggccagccttaaccttgacggctgcaccttgtccttgttccgcgaagacaagcctttcggccccggcaagtttctcggtgactgatatgaaagaccaaaaggacaagcagaccggcgacctgctggccagccctgacgctgtacgccaagcgcgatatgccgagcgcatgaaggccaaagggatgcgtcagcgcaagttctggctgaccgacgacgaatacgaggcgctgcgcgagtgcctggaagaactcagagcggcgcagggcgggggtagtgaccccgccagcgcctaaccaccaactgcctgcaaaggaggcaatcaatggctacccataagcctatcaatattctggaggcgttcgcagcagcgccgccaccgctggactacgttttgcccaacatggtggccggtacggtcggggcgctggtgtcgcccggtggtgccggtaaatccatgctggccctgcaactggccgcacagattgcaggcgggccggatctgctggaggtgggcgaactgcccaccggcccggtgatctacctgcccgccgaagacccgcccaccgccattcatcaccgcctgcacgcccttggggcgcacctcagcgccgaggaacggcaagccgtggctgacggcctgctgatccagccgctgatcggcagcctgcccaacatcatggccccggagtggttcgacggcctcaagcgcgccgccgagggccgccgcctgatggtgctggacacgctgcgccggttccacatcgaggaagaaaacgccagcggccccatggcccaggtcatcggtcgcatggaggccatcgccgccgataccgggtgctctatcgtgttcctgcaccatgccagcaagggcgcggccatgatgggcgcaggcgaccagcagcaggccagccggggcagctcggtactggtcgataacatccgctggcagtcctacctgtcgagcatgaccagcgccgaggccgaggaatggggtgtggacgacgaccagcgccggttcttcgtccgcttcggtgtgagcaaggccaactatggcgcaccgttcgctgatcggtggttcaggcggcatgacggcggggtgctcaagcccgccgtgctggagaggcagcgcaagagcaagggggtgccccgtggtgaagcctaagaacaagcacagcctcagccacgtccggcacgacccggcgcactgtctggcccccggcctgttccgtgccctcaagcggggcgagcgcaagcgcagcaagctggacgtgacgtatgactacggcgacggcaagcggatcgagttcagcggcccggagccgctgggcgctgatgatctgcgcatcctgcaagggctggtggccatggctgggcctaatggcctagtgcttggcccggaacccaagaccgaaggcggacggcagctccggctgttcctggaacccaagtgggaggccgtcaccgctgaatgccatgtggtcaaaggtagctatcgggcgctggcaaaggaaatcggggcagaggtcgatagtggtggggcgctcaagcacatacaggactgcatcgagcgcctttggaaggtatccatcatcgcccagaatggccgcaagcggcaggggtttcggctgctgtcggagtacgccagcgacgaggcggacgggcgcctgtacgtggccctgaaccccttgatcgcgcaggccgtcatgggtggcggccagcatgtgcgcatcagcatggacgaggtgcgggcgctggacagcgaaaccgcccgcctgctgcaccagcggctgtgtggctggatcgaccccggcaaaaccggcaaggcttccatagataccttgtgcggctatgtctggccgtcagaggccagtggttcgaccatgcgcaagcgccgccagcgggtgcgcgaggcgttgccggagctggtcgcgctgggctggacggtaaccgagttcgcggcgggcaagtacgacatcacccggcccaaggcggcaggctgaccccccccactctattgtaaacaagacatttttatcttttatattcaatggcttattttcctgctaattggtaataccatgaaaaataccatgctcagaaaaggcttaacaatattttgaaaaattgcctactgagcgctgccgcacagctccataggccgctttcctggctttgcttccagatgtatgctcttctgctcctgcagctaatggatcaccgcaaacaggttactcgcctggggattccctttcgacccgagcatccgtatgatactcatgctcgattattattattatagaagcccccatgaataaatcgctcatcattttcggcatcgtccatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatctggtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatgggtaccgagctcgaattgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacaatttaaataaataataaaaaagccggattaataatctggctttttatattctctcctaggggcgaattgacattgtgagcggataacaatataatgtgtggatcagcaggacgcactgaccgaattcagaggagaacttaagaatgtctgctgctgctgatagattaaacttaacttccggccacttgaatgctggtagaaagagaagttcctcttctgtttctttgaaggctgccgaaaagcctttcaaggttactgtgattggatctggtaactggggtactactattgccaaggtggttgccgaaaattgtaagggatacccagaagttttcgctccaatagtacaaatgtgggtgttcgaagaagagatcaatggtgaaaaattgactgaaatcataaatactagacatcaaaacgtgaaatacttgcctggcatcactctacccgacaatttggttgctaatccagacttgattgattcagtcaaggatgtcgacatcatcgttttcaacattccacatcaatttttgccccgtatctgtagccaattgaaaggtcatgttgattcacacgtcagagctatctcctgtctaaagggttttgaagttggtgctaaaggtgtccaattgctatcctcttacatcactgaggaactaggtattcaatgtggtgctctatctggtgctaacattgccaccgaagtcgctcaagaacactggtctgaaacaacagttgcttaccacattccaaaggatttcagaggcgagggcaaggacgtcgaccataaggttctaaaggccttgttccacagaccttacttccacgttagtgtcatcgaagatgttgctggtatctccatctgtggtgctttgaagaacgttgttgccttaggttgtggtttcgtcgaaggtctaggctggggtaacaacgcttctgctgccatccaaagagtcggtttgggtgagatcatcagattcggtcaaatgtttttcccagaatctagagaagaaacatactaccaagagtctgctggtgttgctgatttgatcaccacctgcgctggtggtagaaacgtcaaggttgctaggctaatggctacttctggtaaggacgcctgggaatgtgaaaaggagttgttgaatggccaatccgctcaaggtttaattacctgcaaagaagttcacgaatggttggaaacatgtggctctgtcgaagacttcccattatttgaagccgtataccaaatcgtttacaacaactacccaatgaagaacctgccggacatgattgaagaattagatctacatgaagattaggaattcaggagatctacatatgggattgactactaaacctctatctttgaaagttaacgccgctttgttcgacgtcgacggtaccattatcatctctcaaccagccattgctgcattctggagggatttcggtaaggacaaaccttatttcgatgctgaacacgttatccaagtctcgcatggttggagaacgtttgatgccattgctaagttcgctccagactttgccaatgaagagtatgttaacaaattagaagctgaaattccggtcaagtacggtgaaaaatccattgaagtcccaggtgcagttaagctgtgcaacgctttgaacgctctaccaaaagagaaatgggctgtggcaacttccggtacccgtgatatggcacaaaaatggttcgagcatctgggaatcaggagaccaaagtacttcattaccgctaatgatgtcaaacagggtaagcctcatccagaaccatatctgaagggcaggaatggcttaggatatccgatcaatgagcaagacccttccaaatctaaggtagtagtatttgaagacgctccagcaggtattgccgccggaaaagccgccggttgtaagatcattggtattgccactactttcgacttggacttcctaaaggaaaaaggctgtgacatcattgtcaaaaaccacgaatccatcagagttggcggctacaatgccgaaacagacgaagttgaattcatttttgacgactacttatatgctaaggacgatctgttgaaatggtaagaattcaggagcctgcaggaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagaggcctggatcccaagtttactcatatatactttagattgatttaaaacttcatttttaatttaaaaggatctaggtgaagatcctttttgataatctcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaaggatccaggcctgcggccgcacgtacgtacgtacgtacgtatttaaattt

SEQ ID NO: 40 - DNA, 14894 bp, Artificial Sequence
  Synthetic plasmid vector sequence; contains the Psrp promoter linked to dhaB1-3-orfZ-yqhD genes
cgaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataataacgatctgatagagaagggtttgctcgggtcggtggctctggtaacgaccagtatcccgatcccggctggccgtcctggccgccacatgaggcatgttccgcgtccttgcaatactgtgtttacatacagtctatcgcttagcggaaagttcttttaccctcagccgaaatgcctgccgttgctagacattgccagccagtgcccgtcactcccgtactaactgtcacgaacccctgcaataactgtcacgcccccctgcaataactgtcacgaacccctgcaataactgtcacgcccccaaacctgcaaacccagcaggggcgggggctggcggggtgttggaaaaatccatccatgattatctaagaataatccactaggcgcggttatcagcgcccttgtggggcgctgctgcccttgcccaatatgcccggccagaggccggatagctggtctattcgctgcgctaggctacacaccgccccaccgctgcgcggcagggggaaaggcgggcaaagcccgctaaaccccacaccaaaccccgcagaaatacgctggagcgcttttagccgctttagcggcctttccccctacccgaagggtgggggcgcgtgtgcagccccgcagggcctgtctcggtcgatcattcagcccggctcatccttctggcgtggcggcagaccgaacaaggcgcggtcgtggtcgcgttcaaggtacgcatccattgccgccatgagccgatcctccggccactcgctgctgttcaccttggccaaaatcatggcccccaccagcaccttgcgccttgtttcgttcttgcgctcttgctgctgttcccttgcccgcacccgctgaatttcggcattgattcgcgctcgttgttcttcgagcttggccagccgatccgccgccttgttgctccccttaaccatcttgacaccccattgttaatgtgctgtctcgtaggctatcatggaggcacagcggcggcaatcccgaccctactttgtaggggagggcgcacttaccggtttctcttcgagaaactggcctaacggccacccttcgggcggtgcgctctccgagggccattgcatggagccgaaaagcaaaagcaacagcgaggcagcatggcgatttatcaccttacggcgaaaaccggcagcaggtcgggcggccaatcggccagggccaaggccgactacatccagcgcgaaggcaagtatgcccgcgacatggatgaagtcttgcacgccgaatccgggcacatgccggagttcgtcgagcggcccgccgactactgggatgctgccgacctgtatgaacgcgccaatgggcggctgttcaaggaggtcgaatttgccctgccggtcgagctgaccctcgaccagcagaaggcgctggcgtccgagttcgcccagcacctgaccggtgccgagcgcctgccgtatacgctggccatccatgccggtggcggcgagaacccgcactgccacctgatgatctccgagcggatcaatgacggcatcgagcggcccgccgctcagtggttcaagcggtacaacggcaagaccccggagaagggcggggcacagaagaccgaagcgctcaagcccaaggcatggcttgagcagacccgcgaggcatgggccgaccatgccaaccgggcattagagcgggctggccacgacgcccgcattgaccacagaacacttgaggcgcagggcatcgagcgcctgcccggtgttcacctggggccgaacgtggtggagatggaaggccggggcatccgcaccgaccgggcagacgtggccctgaacatcgacaccgccaacgcccagatcatcgacttacaggaataccgggaggcaatagaccatgaacgcaatcgacagagtgaagaaatccagaggcatcaacgagttagcggagcagatcgaaccgctggcccagagcatggcgacactggccgacgaagcccggcaggtcatgagccagacccagcaggccagcgaggcgcaggcggcggagtggctgaaagcccagcgccagacaggggcggcatgggtggagctggccaaagagttgcgggaggtagccgccgaggtgagcagcgccgcgcagagcgcccggagcgcgtcgcgggggtggcactggaagctatggctaaccgtgatgctggcttccatgatgcctacggtggtgctgctgatcgcatcgttgctcttgctcgacctgacgccactgacaaccgaggacggctcgatctggctgcgcttggtggcccgatgaagaacgacaggactttgcaggccataggccgacagctcaaggccatgggctgtgagcgcttcgatatcggcgtcagggacgccaccaccggccagatgatgaaccgggaatggtcagccgccgaagtgctccagaacacgccatggctcaagcggatgaatgcccagggcaatgacgtgtatatcaggcccgccgagcaggagcggcatggtctggtgctggtggacgacctcagcgagtttgacctggatgacatgaaagccgagggccgggagcctgccctggtagtggaaaccagcccgaagaactatcaggcatgggtcaaggtggccgacgccgcaggcggtgaacttcgggggcagattgcccggacgctggccagcgagtacgacgccgacccggccagcgccgacagccgccactatggccgcttggcgggcttcaccaaccgcaaggacaagcacaccacccgcgccggttatcagccgtgggtgctgctgcgtgaatccaagggcaagaccgccaccgctggcccggcgctggtgcagcaggctggccagcagatcgagcaggcccagcggcagcaggagaaggcccgcaggctggccagcctcgaactgcccgagcggcagcttagccgccaccggcgcacggcgctggacgagtaccgcagcgagatggccgggctggtcaagcgcttcggtgatgacctcagcaagtgcgactttatcgccgcgcagaagctggccagccggggccgcagtgccgaggaaatcggcaaggccatggccgaggccagcccagcgctggcagagcgcaagcccggccacgaagcggattacatcgagcgcaccgtcagcaaggtcatgggtctgcccagcgtccagcttgcgcgggccgagctggcacgggcaccggcaccccgccagcgaggcatggacaggggcgggccagatttcagcatgtagtgcttgcgttggtactcacgcctgttatactatgagtactcacgcacagaagggggttttatggaatacgaaaaaagcgcttcagggtcggtctacctgatcaaaagtgacaagggctattggttgcccggtggctttggttatacgtcaaacaaggccgaggctggccgcttttcagtcgctgatatggccagccttaaccttgacggctgcaccttgtccttgttccgcgaagacaagcctttcggccccggcaagtttctcggtgactgatatgaaagaccaaaaggacaagcagaccggcgacctgctggccagccctgacgctgtacgccaagcgcgatatgccgagcgcatgaaggccaaagggatgcgtcagcgcaagttctggctgaccgacgacgaatacgaggcgctgcgcgagtgcctggaagaactcagagcggcgcagggcgggggtagtgaccccgccagcgcctaaccaccaactgcctgcaaaggaggcaatcaatggctacccataagcctatcaatattctggaggcgttcgcagcagcgccgccaccgctggactacgttttgcccaacatggtggccggtacggtcggggcgctggtgtcgcccggtggtgccggtaaatccatgctggccctgcaactggccgcacagattgcaggcgggccggatctgctggaggtgggcgaactgcccaccggcccggtgatctacctgcccgccgaagacccgcccaccgccattcatcaccgcctgcacgcccttggggcgcacctcagcgccgaggaacggcaagccgtggctgacggcctgctgatccagccgctgatcggcagcctgcccaacatcatggccccggagtggttcgacggcctcaagcgcgccgccgagggccgccgcctgatggtgctggacacgctgcgccggttccacatcgaggaagaaaacgccagcggccccatggcccaggtcatcggtcgcatggaggccatcgccgccgataccgggtgctctatcgtgttcctgcaccatgccagcaagggcgcggccatgatgggcgcaggcgaccagcagcaggccagccggggcagctcggtactggtcgataacatccgctggcagtcctacctgtcgagcatgaccagcgccgaggccgaggaatggggtgtggacgacgaccagcgccggttcttcgtccgcttcggtgtgagcaaggccaactatggcgcaccgttcgctgatcggtggttcaggcggcatgacggcggggtgctcaagcccgccgtgctggagaggcagcgcaagagcaagggggtgccccgtggtgaagcctaagaacaagcacagcctcagccacgtccggcacgacccggcgcactgtctggcccccggcctgttccgtgccctcaagcggggcgagcgcaagcgcagcaagctggacgtgacgtatgactacggcgacggcaagcggatcgagttcagcggcccggagccgctgggcgctgatgatctgcgcatcctgcaagggctggtggccatggctgggcctaatggcctagtgcttggcccggaacccaagaccgaaggcggacggcagctccggctgttcctggaacccaagtgggaggccgtcaccgctgaatgccatgtggtcaaaggtagctatcgggcgctggcaaaggaaatcggggcagaggtcgatagtggtggggcgctcaagcacatacaggactgcatcgagcgcctttggaaggtatccatcatcgcccagaatggccgcaagcggcaggggtttcggctgctgtcggagtacgccagcgacgaggcggacgggcgcctgtacgtggccctgaaccccttgatcgcgcaggccgtcatgggtggcggccagcatgtgcgcatcagcatggacgaggtgcgggcgctggacagcgaaaccgcccgcctgctgcaccagcggctgtgtggctggatcgaccccggcaaaaccggcaaggcttccatagataccttgtgcggctatgtctggccgtcagaggccagtggttcgaccatgcgcaagcgccgccagcgggtgcgcgaggcgttgccggagctggtcgcgctgggctggacggtaaccgagttcgcggcgggcaagtacgacatcacccggcccaaggcggcaggctgaccccccccactctattgtaaacaagacatttttatcttttatattcaatggcttattttcctgctaattggtaataccatgaaaaataccatgctcagaaaaggcttaacaatattttgaaaaattgcctactgagcgctgccgcacagctccataggccgctttcctggctttgcttccagatgtatgctcttctgctcctgcagctaatggatcaccgcaaacaggttactcgcctggggattccctttcgacccgagcatccgtatgatactcatgctcgattattattattatagaagcccccatgaataaatcgctcatcattttcggcatcgtccatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatctggtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatgggtaccgagctcgaattgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacaatttaaataaataataaaaaagccggattaataatctggctttttatattctctcctaggggcgaattgacattgtgagcggataacaatataatgtgtggatcagcaggacgcactgaccgaattcaggagcctgcaggatgaaaagatcaaaacgatttgcagtactggcccagcgccccgtcaatcaggacgggctgattggcgagtggcctgaagaggggctgatcgccatggacagcccctttgacccggtctcttcagtaaaagtggacaacggtctgatcgtcgagctggacggcaaacgccgggaccagtttgacatgatcgaccggtttatcgccgattacgcgatcaacgttgagcgcacagagcaggcaatgcgcctggaggcggtggaaatagcccgcatgctggtggatattcacgtcagccgggaggagatcattgccatcactaccgccatcacgccggccaaagcggtcgaggtgatggcgcagatgaacgtggtggagatgatgatggcgctgcagaagatgcgtgcccgccggaccccctccaaccagtgccacgtcaccaatctcaaagataatccggtgcagattgccgctgacgccgccgaggccgggatccgcggcttctcagaacaggagaccacggtcggtatcgcgcgctatgcgccgtttaacgccctggcgctgttggtcggctcgcagtgcggccgtcccggcgtgttgacgcagtgctcggtggaagaggccaccgagctggagctgggcatgcgtggcttaaccagctacgccgagacggtgtcggtctacggcactgaagcggtatttaccgacggcgatgatactccgtggtcaaaggcgttcctcgcctcggcctacgcctcccgcgggttgaaaatgcgctacacctccggcaccggatccgaagcgctgatgggctattcggagagcaagtcgatgctctacctcgaatcgcgctgcatcttcattaccaaaggcgccggggttcaggggctgcaaaacggcgcagtgagctgtatcggcatgaccggcgctgtgccgtcgggcattcgggcggtgctggcggaaaacctgatcgcctctatgctcgacctcgaagtggcgtccgccaacgaccagactttctcccactcggatattcgccgcaccgcgcgcaccctgatgcagatgctgccgggcaccgactttattttctccggctacagcgcggtgccgaactacgacaacatgttcgccggctcgaacttcgatgcggaagattttgatgattacaacattctgcagcgtgacctgatggttgacggcggcctgcgtccggtgaccgaggcggaaaccattgccattcgccagaaagcggcgcgggcgatccaggcggttttccgcgagctggggctgccgccaatcgccgacgaggaggtggaggccgccacctacgcgcacggcagcaacgagatgccgccgcgtaacgtggtggaggatctgagtgcggtggaagagatgatgaagcgcaacatcaccggcctcgatattgtcggcgcgctgagccgcagcggctttgaggatatcgccagcaatattctcaatatgctgcgccagcgggtcaccggcgattacctgcagacctcggccattctcgatcgacagttcgaggtggtgagcgcggtcaacgacatcaatgactatcaggggccgggcaccggctatcgcatctctgccgaacgctgggcggagatcaaaaatattccgggcgtggttcagcctgacaccattgaataaggcggtattcctgtgcaacagacaactcaaattcagccctcttttaccctgaaaacccgcgagggcggggtagcttctgccgatgaacgtgccgatgaagtggtgatcggcgtcggccctgccttcgataaacaccagcatcacactctgatcgatatgccccatggcgcgatcctcaaagagctgattgccggggtggaagaagaggggcttcacgcccgggtggtgcgcattctgcgcacgtccgacgtctcctttatggcctgggatgcggccaacctgagcggctcggggatcggcatcggtatccagtcgaaggggaccacggtcatccatcagcgcgatctgctgccgctcagcaacctggagctgttctcccaggcgccgctgctgacgctggagacctaccggcagattggcaaaaacgccgcgcgctatgcgcgcaaagagtcaccttcgccggtgccggtggtgaacgatcagatggtgcggccgaaatttatggccaaagccgcgctatttcatatcaaagagaccaaacatgtggtgcaggacgccgagcccgtcaccctgcacgtcgacttagtaagggagtgaccatgagcgagaaaaccatgcgcgtgcaggattatccgttagccacccgctgcccggagcatatcctgacgcctaccggcaaaccattgaccgatattaccctcgagaaggtgctctctggcgaggtgggcccgcaggatgtgcggatctcccgccagacccttgagtaccaggcgcagattgccgagcagatgcagcgccatgcggtggcgcgcaatttccgccgcgcggcggagcttatcgccattcctgacgagcgcattctggctatctataacgcgctgcgcccgttccgctcctcgcaggcggagctgctggcgatcgccgacgagctggagcacacctggcatgcgacagtgaatgccgcctttgtccgggagtcggcggaagtgtatcagcagcggcataagctgcgtaaaggaagctaagcggaggtcagcatgccgttaatagccgggattgatatcggcaacgccaccaccgaggtggcgctggcgtccgatgacccgcaggcgagggcgtttgttgccagcgggatcgtcgcgacgacgggcatgaaagggacgcgggacaatatcgccgggaccctcgccgcgctggagcaggccctggcgaaaacaccgtggtcgatgagcgatgtctctcgcatctatcttaacgaagccgcgccggtgattggcgatgtggcgatggagaccatcaccgagaccattatcaccgaatcgaccatgatcggtcataacccgcagacgccgggcggggtgggcgttggcgtggggacgactatcgccctcgggcggctggcgacgctgccggcggcgcagtatgccgaggggtggatcgtactgattgacgacgccgtcgatttccttgacgccgtgtggtggctcaatgaggcgctcgaccgggggatcaacgtggtggcggcgatcctcaaaaaggacgacggcgtgctggtgaacaaccgcctgcgtaaaaccctgccggtggtggatgaagtgacgctgctggagcaggtccccgagggggtaatggcggcggtggaagtggccgcgccgggccaggtggtgcggatcctgtcgaatccctacgggatcgccaccttcttcgggctaagcccggaagagacccaggccatcgtccccatcgcccgcgccctgattggcaaccgttcagcggtggtgctcaagaccccgcagggggatgtgcagtcgcgggtgatcccggcgggcaacctctacattagcggcgaaaagcgccgcggagaggccgatgtcgccgagggcgcggaagccatcatgcaggcgatgagcgcctgcgctccggtacgcgacatccgcggcgaaccgggcacccacgccggcggcatgcttgagcgggtgcgcaaggtaatggcgtccctgaccggccatgagatgagcgcgatatacatccaggatctgctggcggtggatacgtttattccgcgcaaggtgcagggcgggatggccggcgagtgcgccatggagaatgccgtcgggatggcggcgatggtgaaagcggatcgtctgcaaatgcaggttatcgcccgcgaactgagcgcccgactgcagaccgaggtggtggtgggcggcgtggaggccaacatggccatcgccggggcgttaaccactcccggctgtgcggcgccgctggcgatcctcgacctcggcgccggctcgacggatgcggcgatcgtcaacgcggaggggcagataacggcggtccatctcgccggggcggggaatatggtcagcctgttgattaaaaccgagctgggcctcgaggatctttcgctggcggaagcgataaaaaaatacccgctggccaaagtggaaagcctgttcagtattcgtcacgagaatggcgcggtggagttctttcgggaagccctcagcccggcggtgttcgccaaagtggtgtacatcaaggagggcgaactggtgccgatcgataacgccagcccgctggaaaaaattcgtctcgtgcgccggcaggcgaaagagaaagtgtttgtcaccaactgcctgcgcgcgctgcgccaggtctcacccggcggttccattcgcgatatcgcctttgtggtgctggtgggcggctcatcgctggactttgagatcccgcagcttatcacggaagccttgtcgcactatggcgtggtcgccgggcagggcaatattcggggaacagaagggccgcgcaatgcggtcgccaccgggctgctactggccggtcaggcgaattaaggcgcgccaagaggagagctagcgatgaacaactttaatctgcacaccccaacccgcattctgtttggtaaaggcgcaatcgctggtttacgcgaacaaattcctcacgatgctcgcgtattgattacctacggcggcggcagcgtgaaaaaaaccggcgttctcgatcaagttctggatgccctgaaaggcatggacgtgctggaatttggcggtattgagccaaacccggcttatgaaacgctgatgaacgccgtgaaactggttcgcgaacagaaagtgactttcctgctggcggttggcggcggttctgtactggacggcaccaaatttatcgccgcagcggctaactatccggaaaatatcgatccgtggcacattctgcaaacgggcggtaaagagattaaaagcgccatcccgatgggctgtgtgctgacgctgccagcaaccggttcagaatccaacgcaggcgcggtgatctcccgtaaaaccacaggcgacaagcaggcgttccattctgcccatgttcagccggtatttgccgtgctcgatccggtttatacctacaccctgccgccgcgtcaggtggctaacggcgtagtggacgcctttgtacacaccgtggaacagtatgttaccaaaccggttgatgccaaaattcaggaccgtttcgcagaaggcattttgctgacgctaatcgaagatggtccgaaagccctgaaagagccagaaaactacgatgtgcgcgccaacgtcatgtgggcggcgactcaggcgctgaacggtttgattggcgctggcgtaccgcaggactgggcaacgcatatgctgggccacgaactgactgcgatgcacggtctggatcacgcgcaaacactggctatcgtcctgcctgcactgtggaatgaaaaacgcgataccaagcgcgctaagctgctgcaatatgctgaacgcgtctggaacatcactgaaggttccgatgatgagcgtattgacgccgcgattgccgcaacccgcaatttctttgagcaattaggcgtgccgacccacctctccgactacggtctggacggcagctccatcccggctttgctgaaaaaactggaagagcacggcatgacccaactgggcgaaaatcatgacattacgttggatgtcagccgccgtatatacgaagccgcccgctaagcggcgcgcctgcaggaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagaggcctggatcccaagtttactcatatatactttagattgatttaaaacttcatttttaatttaaaaggatctaggtgaagatcctttttgataatctcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaaggatccaggcctgcggccgcacgtacgtacgtacgtacgtatttaaattt

SEQ ID NO: 41 - DNA, 16784 bp, Artificial Sequence
  Synthetic plasmid vector sequence; contains the pnblA7120 promoter linked to the genes DAR1-GPP2-dhaB1-3-orfZ-orf2b-yqhD
aacgatctgatagagaagggtttgctcgggtcggtggctctggtaacgaccagtatcccgatcccggctggccgtcctggccgccacatgaggcatgttccgcgtccttgcaatactgtgtttacatacagtctatcgcttagcggaaagttcttttaccctcagccgaaatgcctgccgttgctagacattgccagccagtgcccgtcactcccgtactaactgtcacgaacccctgcaataactgtcacgcccccctgcaataactgtcacgaacccctgcaataactgtcacgcccccaaacctgcaaacccagcaggggcgggggctggcggggtgttggaaaaatccatccatgattatctaagaataatccactaggcgcggttatcagcgcccttgtggggcgctgctgcccttgcccaatatgcccggccagaggccggatagctggtctattcgctgcgctaggctacacaccgccccaccgctgcgcggcagggggaaaggcgggcaaagcccgctaaaccccacaccaaaccccgcagaaatacgctggagcgcttttagccgctttagcggcctttccccctacccgaagggtgggggcgcgtgtgcagccccgcagggcctgtctcggtcgatcattcagcccggctcatccttctggcgtggcggcagaccgaacaaggcgcggtcgtggtcgcgttcaaggtacgcatccattgccgccatgagccgatcctccggccactcgctgctgttcaccttggccaaaatcatggcccccaccagcaccttgcgccttgtttcgttcttgcgctcttgctgctgttcccttgcccgcacccgctgaatttcggcattgattcgcgctcgttgttcttcgagcttggccagccgatccgccgccttgttgctccccttaaccatcttgacaccccattgttaatgtgctgtctcgtaggctatcatggaggcacagcggcggcaatcccgaccctactttgtaggggagggcgcacttaccggtttctcttcgagaaactggcctaacggccacccttcgggcggtgcgctctccgagggccattgcatggagccgaaaagcaaaagcaacagcgaggcagcatggcgatttatcaccttacggcgaaaaccggcagcaggtcgggcggccaatcggccagggccaaggccgactacatccagcgcgaaggcaagtatgcccgcgacatggatgaagtcttgcacgccgaatccgggcacatgccggagttcgtcgagcggcccgccgactactgggatgctgccgacctgtatgaacgcgccaatgggcggctgttcaaggaggtcgaatttgccctgccggtcgagctgaccctcgaccagcagaaggcgctggcgtccgagttcgcccagcacctgaccggtgccgagcgcctgccgtatacgctggccatccatgccggtggcggcgagaacccgcactgccacctgatgatctccgagcggatcaatgacggcatcgagcggcccgccgctcagtggttcaagcggtacaacggcaagaccccggagaagggcggggcacagaagaccgaagcgctcaagcccaaggcatggcttgagcagacccgcgaggcatgggccgaccatgccaaccgggcattagagcgggctggccacgacgcccgcattgaccacagaacacttgaggcgcagggcatcgagcgcctgcccggtgttcacctggggccgaacgtggtggagatggaaggccggggcatccgcaccgaccgggcagacgtggccctgaacatcgacaccgccaacgcccagatcatcgacttacaggaataccgggaggcaatagaccatgaacgcaatcgacagagtgaagaaatccagaggcatcaacgagttagcggagcagatcgaaccgctggcccagagcatggcgacactggccgacgaagcccggcaggtcatgagccagacccagcaggccagcgaggcgcaggcggcggagtggctgaaagcccagcgccagacaggggcggcatgggtggagctggccaaagagttgcgggaggtagccgccgaggtgagcagcgccgcgcagagcgcccggagcgcgtcgcgggggtggcactggaagctatggctaaccgtgatgctggcttccatgatgcctacggtggtgctgctgatcgcatcgttgctcttgctcgacctgacgccactgacaaccgaggacggctcgatctggctgcgcttggtggcccgatgaagaacgacaggactttgcaggccataggccgacagctcaaggccatgggctgtgagcgcttcgatatcggcgtcagggacgccaccaccggccagatgatgaaccgggaatggtcagccgccgaagtgctccagaacacgccatggctcaagcggatgaatgcccagggcaatgacgtgtatatcaggcccgccgagcaggagcggcatggtctggtgctggtggacgacctcagcgagtttgacctggatgacatgaaagccgagggccgggagcctgccctggtagtggaaaccagcccgaagaactatcaggcatgggtcaaggtggccgacgccgcaggcggtgaacttcgggggcagattgcccggacgctggccagcgagtacgacgccgacccggccagcgccgacagccgccactatggccgcttggcgggcttcaccaaccgcaaggacaagcacaccacccgcgccggttatcagccgtgggtgctgctgcgtgaatccaagggcaagaccgccaccgctggcccggcgctggtgcagcaggctggccagcagatcgagcaggcccagcggcagcaggagaaggcccgcaggctggccagcctcgaactgcccgagcggcagcttagccgccaccggcgcacggcgctggacgagtaccgcagcgagatggccgggctggtcaagcgcttcggtgatgacctcagcaagtgcgactttatcgccgcgcagaagctggccagccggggccgcagtgccgaggaaatcggcaaggccatggccgaggccagcccagcgctggcagagcgcaagcccggccacgaagcggattacatcgagcgcaccgtcagcaaggtcatgggtctgcccagcgtccagcttgcgcgggccgagctggcacgggcaccggcaccccgccagcgaggcatggacaggggcgggccagatttcagcatgtagtgcttgcgttggtactcacgcctgttatactatgagtactcacgcacagaagggggttttatggaatacgaaaaaagcgcttcagggtcggtctacctgatcaaaagtgacaagggctattggttgcccggtggctttggttatacgtcaaacaaggccgaggctggccgcttttcagtcgctgatatggccagccttaaccttgacggctgcaccttgtccttgttccgcgaagacaagcctttcggccccggcaagtttctcggtgactgatatgaaagaccaaaaggacaagcagaccggcgacctgctggccagccctgacgctgtacgccaagcgcgatatgccgagcgcatgaaggccaaagggatgcgtcagcgcaagttctggctgaccgacgacgaatacgaggcgctgcgcgagtgcctggaagaactcagagcggcgcagggcgggggtagtgaccccgccagcgcctaaccaccaactgcctgcaaaggaggcaatcaatggctacccataagcctatcaatattctggaggcgttcgcagcagcgccgccaccgctggactacgttttgcccaacatggtggccggtacggtcggggcgctggtgtcgcccggtggtgccggtaaatccatgctggccctgcaactggccgcacagattgcaggcgggccggatctgctggaggtgggcgaactgcccaccggcccggtgatctacctgcccgccgaagacccgcccaccgccattcatcaccgcctgcacgcccttggggcgcacctcagcgccgaggaacggcaagccgtggctgacggcctgctgatccagccgctgatcggcagcctgcccaacatcatggccccggagtggttcgacggcctcaagcgcgccgccgagggccgccgcctgatggtgctggacacgctgcgccggttccacatcgaggaagaaaacgccagcggccccatggcccaggtcatcggtcgcatggaggccatcgccgccgataccgggtgctctatcgtgttcctgcaccatgccagcaagggcgcggccatgatgggcgcaggcgaccagcagcaggccagccggggcagctcggtactggtcgataacatccgctggcagtcctacctgtcgagcatgaccagcgccgaggccgaggaatggggtgtggacgacgaccagcgccggttcttcgtccgcttcggtgtgagcaaggccaactatggcgcaccgttcgctgatcggtggttcaggcggcatgacggcggggtgctcaagcccgccgtgctggagaggcagcgcaagagcaagggggtgccccgtggtgaagcctaagaacaagcacagcctcagccacgtccggcacgacccggcgcactgtctggcccccggcctgttccgtgccctcaagcggggcgagcgcaagcgcagcaagctggacgtgacgtatgactacggcgacggcaagcggatcgagttcagcggcccggagccgctgggcgctgatgatctgcgcatcctgcaagggctggtggccatggctgggcctaatggcctagtgcttggcccggaacccaagaccgaaggcggacggcagctccggctgttcctggaacccaagtgggaggccgtcaccgctgaatgccatgtggtcaaaggtagctatcgggcgctggcaaaggaaatcggggcagaggtcgatagtggtggggcgctcaagcacatacaggactgcatcgagcgcctttggaaggtatccatcatcgcccagaatggccgcaagcggcaggggtttcggctgctgtcggagtacgccagcgacgaggcggacgggcgcctgtacgtggccctgaaccccttgatcgcgcaggccgtcatgggtggcggccagcatgtgcgcatcagcatggacgaggtgcgggcgctggacagcgaaaccgcccgcctgctgcaccagcggctgtgtggctggatcgaccccggcaaaaccggcaaggcttccatagataccttgtgcggctatgtctggccgtcagaggccagtggttcgaccatgcgcaagcgccgccagcgggtgcgcgaggcgttgccggagctggtcgcgctgggctggacggtaaccgagttcgcggcgggcaagtacgacatcacccggcccaaggcggcaggctgaccccccccactctattgtaaacaagacatttttatcttttatattcaatggcttattttcctgctaattggtaataccatgaaaaataccatgctcagaaaaggcttaacaatattttgaaaaattgcctactgagcgctgccgcacagctccataggccgctttcctggctttgcttccagatgtatgctcttctgctcctgcagctaatggatcaccgcaaacaggttactcgcctggggattccctttcgacccgagcatccgtatgatactcatgctcgattattattattatagaagcccccatgaataaatcgctcatcattttcggcatcgtccctgttaacggatccagagaatataaaaagccagattattaatccggcttttttattatttgccgtagagctattcactttaggtttaggatgaaaaaaaataaaaaaggggacctctagggtccccaattaattagtaatataatctattaaaggtcattcaaaaggtcatccaccggatcagcttagtaaagccctcgctagattttaatgcggatgttgcgattacttcgccaactattgcgataacaagaaaaagccagcctttcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccagcttctgtatggaacgggcatgcggatcagtgagggtttgcaactgcgggtcaaggatctggatttcgatcacggcacgatcatcgtgcgggagggcaagggctccaaggatcgggccttgatgttacccgagagcttggcacccagcctgcgcgagcaggggaattgatccggtggatgaccttttgaatgacctttaatagattatattactaattaattggggaccctagaggtccccttttttattttactgcgatgagtggcagggcggggcgtaattttttttacgctttacttacgtacttaattcttaaagtatgggcaatcaattggtcgacgataacatcaccgtcgttatcgtcgctttagaataacgttcccaaaatagctcatttccaactggcaactcacaaccaaaaaccgcatttttagtaaatatactcagcaatttgttcaacctgagcatttttcccatttgcaacttgatacaaatatttttagcagcaaattttcctactgccagcttagtttacataaattttgtctgttgacatcttgcacacaataaggtatggcgcatataatgcgatattactaccattaatttactacctagtcattaacgtctcccgccagagaacagttttgaataggtagtcaattttaggtattgaacctgctgtaaatttattaaatcgatgaatttccccgaaatctgctctagcagacttgggttatataccagtaggctcaggtgcaaaacaacaaagcacaaattttacccattaaggatataggcaatctgtcaaatagttgttatctttcttaatacagaggaataatcaacaatatggggcaggtactaactaaagtcctatgcctgtggggcttctgtaaccgacataacctttacgcgttgtcttttaggagtctgttatgaacggtaccatgaattcatgtctgctgctgctgatagattaaacttaacttccggccacttgaatgctggtagaaagagaagttcctcttctgtttctttgaaggctgccgaaaagcctttcaaggttactgtgattggatctggtaactggggtactactattgccaaggtggttgccgaaaattgtaagggatacccagaagttttcgctccaatagtacaaatgtgggtgttcgaagaagagatcaatggtgaaaaattgactgaaatcataaatactagacatcaaaacgtgaaatacttgcctggcatcactctacccgacaatttggttgctaatccagacttgattgattcagtcaaggatgtcgacatcatcgttttcaacattccacatcaatttttgccccgtatctgtagccaattgaaaggtcatgttgattcacacgtcagagctatctcctgtctaaagggttttgaagttggtgctaaaggtgtccaattgctatcctcttacatcactgaggaactaggtattcaatgtggtgctctatctggtgctaacattgccaccgaagtcgctcaagaacactggtctgaaacaacagttgcttaccacattccaaaggatttcagaggcgagggcaaggacgtcgaccataaggttctaaaggccttgttccacagaccttacttccacgttagtgtcatcgaagatgttgctggtatctccatctgtggtgctttgaagaacgttgttgccttaggttgtggtttcgtcgaaggtctaggctggggtaacaacgcttctgctgccatccaaagagtcggtttgggtgagatcatcagattcggtcaaatgtttttcccagaatctagagaagaaacatactaccaagagtctgctggtgttgctgatttgatcaccacctgcgctggtggtagaaacgtcaaggttgctaggctaatggctacttctggtaaggacgcctgggaatgtgaaaaggagttgttgaatggccaatccgctcaaggtttaattacctgcaaagaagttcacgaatggttggaaacatgtggctctgtcgaagacttcccattatttgaagccgtataccaaatcgtttacaacaactacccaatgaagaacctgccggacatgattgaagaattagatctacatgaagattaggaattcaggagatctacatatgggattgactactaaacctctatctttgaaagttaacgccgctttgttcgacgtcgacggtaccattatcatctctcaaccagccattgctgcattctggagggatttcggtaaggacaaaccttatttcgatgctgaacacgttatccaagtctcgcatggttggagaacgtttgatgccattgctaagttcgctccagactttgccaatgaagagtatgttaacaaattagaagctgaaattccggtcaagtacggtgaaaaatccattgaagtcccaggtgcagttaagctgtgcaacgctttgaacgctctaccaaaagagaaatgggctgtggcaacttccggtacccgtgatatggcacaaaaatggttcgagcatctgggaatcaggagaccaaagtacttcattaccgctaatgatgtcaaacagggtaagcctcatccagaaccatatctgaagggcaggaatggcttaggatatccgatcaatgagcaagacccttccaaatctaaggtagtagtatttgaagacgctccagcaggtattgccgccggaaaagccgccggttgtaagatcattggtattgccactactttcgacttggacttcctaaaggaaaaaggctgtgacatcattgtcaaaaaccacgaatccatcagagttggcggctacaatgccgaaacagacgaagttgaattcatttttgacgactacttatatgctaaggacgatctgttgaaatggtaagaattcaggagcctgcaggatgaaaagatcaaaacgatttgcagtactggcccagcgccccgtcaatcaggacgggctgattggcgagtggcctgaagaggggctgatcgccatggacagcccctttgacccggtctcttcagtaaaagtggacaacggtctgatcgtcgagctggacggcaaacgccgggaccagtttgacatgatcgaccggtttatcgccgattacgcgatcaacgttgagcgcacagagcaggcaatgcgcctggaggcggtggaaatagcccgcatgctggtggatattcacgtcagccgggaggagatcattgccatcactaccgccatcacgccggccaaagcggtcgaggtgatggcgcagatgaacgtggtggagatgatgatggcgctgcagaagatgcgtgcccgccggaccccctccaaccagtgccacgtcaccaatctcaaagataatccggtgcagattgccgctgacgccgccgaggccgggatccgcggcttctcagaacaggagaccacggtcggtatcgcgcgctatgcgccgtttaacgccctggcgctgttggtcggctcgcagtgcggccgtcccggcgtgttgacgcagtgctcggtggaagaggccaccgagctggagctgggcatgcgtggcttaaccagctacgccgagacggtgtcggtctacggcactgaagcggtatttaccgacggcgatgatactccgtggtcaaaggcgttcctcgcctcggcctacgcctcccgcgggttgaaaatgcgctacacctccggcaccggatccgaagcgctgatgggctattcggagagcaagtcgatgctctacctcgaatcgcgctgcatcttcattaccaaaggcgccggggttcaggggctgcaaaacggcgcagtgagctgtatcggcatgaccggcgctgtgccgtcgggcattcgggcggtgctggcggaaaacctgatcgcctctatgctcgacctcgaagtggcgtccgccaacgaccagactttctcccactcggatattcgccgcaccgcgcgcaccctgatgcagatgctgccgggcaccgactttattttctccggctacagcgcggtgccgaactacgacaacatgttcgccggctcgaacttcgatgcggaagattttgatgattacaacattctgcagcgtgacctgatggttgacggcggcctgcgtccggtgaccgaggcggaaaccattgccattcgccagaaagcggcgcgggcgatccaggcggttttccgcgagctggggctgccgccaatcgccgacgaggaggtggaggccgccacctacgcgcacggcagcaacgagatgccgccgcgtaacgtggtggaggatctgagtgcggtggaagagatgatgaagcgcaacatcaccggcctcgatattgtcggcgcgctgagccgcagcggctttgaggatatcgccagcaatattctcaatatgctgcgccagcgggtcaccggcgattacctgcagacctcggccattctcgatcgacagttcgaggtggtgagcgcggtcaacgacatcaatgactatcaggggccgggcaccggctatcgcatctctgccgaacgctgggcggagatcaaaaatattccgggcgtggttcagcctgacaccattgaataaggcggtattcctgtgcaacagacaactcaaattcagccctcttttaccctgaaaacccgcgagggcggggtagcttctgccgatgaacgtgccgatgaagtggtgatcggcgtcggccctgccttcgataaacaccagcatcacactctgatcgatatgccccatggcgcgatcctcaaagagctgattgccggggtggaagaagaggggcttcacgcccgggtggtgcgcattctgcgcacgtccgacgtctcctttatggcctgggatgcggccaacctgagcggctcggggatcggcatcggtatccagtcgaaggggaccacggtcatccatcagcgcgatctgctgccgctcagcaacctggagctgttctcccaggcgccgctgctgacgctggagacctaccggcagattggcaaaaacgccgcgcgctatgcgcgcaaagagtcaccttcgccggtgccggtggtgaacgatcagatggtgcggccgaaatttatggccaaagccgcgctatttcatatcaaagagaccaaacatgtggtgcaggacgccgagcccgtcaccctgcacgtcgacttagtaagggagtgaccatgagcgagaaaaccatgcgcgtgcaggattatccgttagccacccgctgcccggagcatatcctgacgcctaccggcaaaccattgaccgatattaccctcgagaaggtgctctctggcgaggtgggcccgcaggatgtgcggatctcccgccagacccttgagtaccaggcgcagattgccgagcagatgcagcgccatgcggtggcgcgcaatttccgccgcgcggcggagcttatcgccattcctgacgagcgcattctggctatctataacgcgctgcgcccgttccgctcctcgcaggcggagctgctggcgatcgccgacgagctggagcacacctggcatgcgacagtgaatgccgcctttgtccgggagtcggcggaagtgtatcagcagcggcataagctgcgtaaaggaagctaagcggaggtcagcatgccgttaatagccgggattgatatcggcaacgccaccaccgaggtggcgctggcgtccgatgacccgcaggcgagggcgtttgttgccagcgggatcgtcgcgacgacgggcatgaaagggacgcgggacaatatcgccgggaccctcgccgcgctggagcaggccctggcgaaaacaccgtggtcgatgagcgatgtctctcgcatctatcttaacgaagccgcgccggtgattggcgatgtggcgatggagaccatcaccgagaccattatcaccgaatcgaccatgatcggtcataacccgcagacgccgggcggggtgggcgttggcgtggggacgactatcgccctcgggcggctggcgacgctgccggcggcgcagtatgccgaggggtggatcgtactgattgacgacgccgtcgatttccttgacgccgtgtggtggctcaatgaggcgctcgaccgggggatcaacgtggtggcggcgatcctcaaaaaggacgacggcgtgctggtgaacaaccgcctgcgtaaaaccctgccggtggtggatgaagtgacgctgctggagcaggtccccgagggggtaatggcggcggtggaagtggccgcgccgggccaggtggtgcggatcctgtcgaatccctacgggatcgccaccttcttcgggctaagcccggaagagacccaggccatcgtccccatcgcccgcgccctgattggcaaccgttcagcggtggtgctcaagaccccgcagggggatgtgcagtcgcgggtgatcccggcgggcaacctctacattagcggcgaaaagcgccgcggagaggccgatgtcgccgagggcgcggaagccatcatgcaggcgatgagcgcctgcgctccggtacgcgacatccgcggcgaaccgggcacccacgccggcggcatgcttgagcgggtgcgcaaggtaatggcgtccctgaccggccatgagatgagcgcgatatacatccaggatctgctggcggtggatacgtttattccgcgcaaggtgcagggcgggatggccggcgagtgcgccatggagaatgccgtcgggatggcggcgatggtgaaagcggatcgtctgcaaatgcaggttatcgcccgcgaactgagcgcccgactgcagaccgaggtggtggtgggcggcgtggaggccaacatggccatcgccggggcgttaaccactcccggctgtgcggcgccgctggcgatcctcgacctcggcgccggctcgacggatgcggcgatcgtcaacgcggaggggcagataacggcggtccatctcgccggggcggggaatatggtcagcctgttgattaaaaccgagctgggcctcgaggatctttcgctggcggaagcgataaaaaaatacccgctggccaaagtggaaagcctgttcagtattcgtcacgagaatggcgcggtggagttctttcgggaagccctcagcccggcggtgttcgccaaagtggtgtacatcaaggagggcgaactggtgccgatcgataacgccagcccgctggaaaaaattcgtctcgtgcgccggcaggcgaaagagaaagtgtttgtcaccaactgcctgcgcgcgctgcgccaggtctcacccggcggttccattcgcgatatcgcctttgtggtgctggtgggcggctcatcgctggactttgagatcccgcagcttatcacggaagccttgtcgcactatggcgtggtcgccgggcagggcaatattcggggaacagaagggccgcgcaatgcggtcgccaccgggctgctactggccggtcaggcgaattaaggcgcgccaagaggagaactagtaatgtcgctttcaccgccaggcgtacgcctgttttacgatccgcgcgggcaccatgccggcgccatcaatgagctgtgctgggggctggaggagcagggggtcccctgccagaccataacctatgacggaggcggtgacgccgctgcgctgggcgccctggcggccagaagctcgcccctgcgggtgggtatcgggctcagcgcgtccggcgagatagccctcactcatgcccagctgccggcggacgcgccgctggctaccggacacgtcaccgatagcgacgatcatctgcgtacgctcggcgccaacgccgggcagctggttaaagtcctgccgttaagtgagagaaactgaggcgcgccaagaggagagctagcgatgaacaactttaatctgcacaccccaacccgcattctgtttggtaaaggcgcaatcgctggtttacgcgaacaaattcctcacgatgctcgcgtattgattacctacggcggcggcagcgtgaaaaaaaccggcgttctcgatcaagttctggatgccctgaaaggcatggacgtgctggaatttggcggtattgagccaaacccggcttatgaaacgctgatgaacgccgtgaaactggttcgcgaacagaaagtgactttcctgctggcggttggcggcggttctgtactggacggcaccaaatttatcgccgcagcggctaactatccggaaaatatcgatccgtggcacattctgcaaacgggcggtaaagagattaaaagcgccatcccgatgggctgtgtgctgacgctgccagcaaccggttcagaatccaacgcaggcgcggtgatctcccgtaaaaccacaggcgacaagcaggcgttccattctgcccatgttcagccggtatttgccgtgctcgatccggtttatacctacaccctgccgccgcgtcaggtggctaacggcgtagtggacgcctttgtacacaccgtggaacagtatgttaccaaaccggttgatgccaaaattcaggaccgtttcgcagaaggcattttgctgacgctaatcgaagatggtccgaaagccctgaaagagccagaaaactacgatgtgcgcgccaacgtcatgtgggcggcgactcaggcgctgaacggtttgattggcgctggcgtaccgcaggactgggcaacgcatatgctgggccacgaactgactgcgatgcacggtctggatcacgcgcaaacactggctatcgtcctgcctgcactgtggaatgaaaaacgcgataccaagcgcgctaagctgctgcaatatgctgaacgcgtctggaacatcactgaaggttccgatgatgagcgtattgacgccgcgattgccgcaacccgcaatttctttgagcaattaggcgtgccgacccacctctccgactacggtctggacggcagctccatcccggctttgctgaaaaaactggaagagcacggcatgacccaactgggcgaaaatcatgacattacgttggatgtcagccgccgtatatacgaagccgcccgctaagcggcgcgcctgcatcgagcggccgctagatctgcatgctctagatttaaatgatatcccggcttatcggtcagtttcacctgatttacgtaaaaacccgcttcggcgggtttttgcttttggaggggcagaaagatgaatgactgtccacgacgctatacccaaaagaaagctagcgttaacagg